(19) United States
(12) Patent Application Publication
(10) Pub. No.: US 2019/0321012 A1
(43) Pub. Date: Oct. 24, 2019

(54) DISPOSABLE NEEDLE BIOPSY DEVICE AND NEEDLE STRUCTURE OF CUTTING BIOPSY APPARATUS

(71) Applicant: FLOWER MEDICAL CO., LTD., Gyeonggi-do (KR)

(72) Inventor: Hyung Jin KO, Gyeonggi-do (KR)

(73) Assignee: FLOWER MEDICAL CO., LTD., Gyeonggi-do (KR)

(21) Appl. No.: 16/475,741

(22) PCT Filed: Jan. 3, 2017
(86) PCT No.: PCT/KR2018/000123

§ 371 Date: Jul. 3, 2019

(30) Foreign Application Priority Data
Jan. 3, 2017 (KR) 10-2017-0000555
Feb. 3, 2017 (KR) 10-2017-0015291

(51) Int. Cl. A61B 10/02

(52) U.S. Cl. CPC A61B 10/0275; A61B 2017/0023

(57) ABSTRACT

Provided is a disposable cutting biopsy device including a housing formed in a tube shape and having a sliding space therein, a needle set disposed in the housing through a front portion hole of the housing and comprising an inner needle having a tissue extraction recess to extract a tissue and an outer needle having a blade formed at a leading end to cut the tissue accommodated in the tissue extraction recess and performing a reciprocating motion along an outer circumference of the inner needle in a form of surrounding the inner needle, a sliding block connected to the outer needle and disposed to perform a reciprocating motion back and forth in the sliding space, a loading unit connected to the sliding block and shooting the sliding block, and a loading handle disposed to be connected to the loading unit, in which, when the sliding block moves backward, a first negative pressure space is formed in a front portion of the sliding space, and when the sliding block moves forward, a second negative pressure space is formed in a rear portion of the sliding space.

### CROSS-REFERENCE TO RELATED APPLICATIONS

This application claims priority to and the benefit of Korean Patent Application No. 10-2018-0073724, filed on Jun. 27, 2018, which is hereby incorporated in its entirety by reference for all purposes as set forth herein.

### TECHNICAL FIELD

One embodiment of the present disclosure relates to a disposable needle biopsy device, and more particularly, to a disposable needle biopsy device capable of extracting a tissue sample by cutting a target biological tissue. Another embodiment of the present disclosure relates to a biopsy device of a biological tissue, and more particularly, to a new structure and shape of an inner needle and an outer needle of a biological tissue biopsy device for extracting a biological tissue by allowing a biopsy needle including an inner needle and an outer needle to approach the biological tissue by controlling a main body, accommodating a biological tissue sample in a tissue extraction recess of the inner needle as the operator manipulates a movement of the outer needle, and cutting an accommodated sample from the biological tissue and sealing the tissue extraction recess of the inner needle, thereby extracting a biological tissue.

### BACKGROUND ART

In general, biopsy tests are carried out to extract a patient's tissue for diagnosis and treatments of diseases. Biopsy is a test to identify the existence or the diffusion pattern of a disease by extracting a part of a biological tissue, and the extracted tissue is used for examination and an inflammatory diseases test.

As a device for a biopsy test, for a disposable test device, a disposable cutting biopsy device that extracts a tissue by shooting a needle set including an inner needle and an outer needle toward a specimen and extracting a specimen tissue has been widely used.

FIG. 1 illustrates a cutting biopsy device according to the related art. Referring to FIG. 1, according to a tissue extraction mechanism of a disposable cutting biopsy device according to the related art, an inner needle having a tissue extraction recess and an outer needle having a cutting blade are inserted into a living body and then the inner needle and the outer needle are sequentially shot toward a specimen by means of a shooting device. The inner needle of a needle set inserted into a target tissue spaced apart by a certain distance therefrom (step1) is first strongly forward shot by elasticity of a spring (step2), and then the outer needle is strongly forward shot by the elasticity of the spring (step3), thereby cutting the target specimen.

The above needle extraction method may need to extract a large amount of a tissue by repeating an operation many times to secure test accuracy. Accordingly, improving extraction yield and the securing of safety are important.

To improve an extraction yield, a negative pressure may be used. Technology to introduce a vacuum source for generating vacuum has been introduced to use the negative pressure.

The negative pressure technology has not been used in a disposable cutting biopsy device due to cost, and has been mostly applied to dedicated equipment having a separate vacuum source.

Korean Patent No. 10-1649713 and U.S. Pat. No. 8,679,032 B2 disclose the operation mechanism of a negative pressure. As the mechanism makes a needle set thick and requires a configuration of a complicated apparatus to install a vacuum source, it is difficult to be applied to a disposable cutting biopsy device.

Most disposable cutting biopsy devices are provided with a block for fixing each of an inner needle and an outer needle to drive the mechanism, and have a structure of driving the inner needle and the outer needle by using two compression springs connected thereto. Korean Patent No. 10-1551311 and Korean Patent No. 10-1463867 disclose such structures.

The structures have a forward shot type in common and are inefficient because a negative pressure is applied after the inner needle arrives at a tissue due to the characteristics of the operation mechanism of a forward shot. Furthermore, when there is an error predicting an aiming distance, the shooting of the inner needle cannot be stopped in the middle, thereby generating a fatal risk.

### DESCRIPTION OF EMBODIMENTS

### Technical Problem

To address the above matters, provided is a disposable cutting biopsy device which may improve a tissue extraction yield by implementing a negative pressure mechanism that generates a negative pressure by only an operation of a simple device without a separate negative pressure generation apparatus and may be applied to a process of first accommodating a tissue to be extracted and completing tissue cutting once the tissue is accommodated.

Provide is a disposable cutting biopsy device that matches the purpose of a disposable biopsy device by simplifying the configuration of a device.

Provide is a disposable cutting biopsy device that secures precision, convenience, and safety.

### Solution to Problem

According to an aspect of the present disclosure, a disposable cutting biopsy device includes a housing formed in a tube shape and having a sliding space therein, a needle set disposed in the housing through a front portion hole of the housing and comprising an inner needle having a tissue extraction recess to extract a tissue and an outer needle having a blade formed at a leading end to cut the tissue accommodated in the tissue extraction recess and performing a reciprocating motion along an outer circumference of the inner needle in a form of surrounding the inner needle, a sliding block connected to the outer needle and disposed to perform a reciprocating motion back and forth in the sliding space, a loading unit connected to the sliding block and shooting the sliding block, and a loading handle disposed to be connected to the loading unit.

When the sliding block moves backward, a first negative pressure space is formed in a front portion of the sliding space, and when the sliding block moves forward, a second negative pressure space is formed in a rear portion of the sliding space

A negative pressure from the first negative pressure space and the second negative pressure space may be applied to the tissue extraction recess.

The housing may further include a first positive pressure valve formed in a front portion of the housing to remove a positive pressure formed in the first negative pressure space as the sliding block moves forward, and a second positive pressure valve formed in a rear portion of the housing to remove a positive pressure formed in the second negative pressure space as the sliding block moves backward.

When the sliding block moves backward, the first positive pressure valve may be closed and a negative pressure is formed in the first negative pressure space, and the second positive pressure valve may be opened and a positive pressure of the second negative pressure space may be removed, and when the sliding block moves forward, the first positive pressure valve may be opened and a positive pressure of the first negative pressure space may be removed, and the second positive pressure valve may be closed and a negative pressure may be formed in the second negative pressure space.

The inner needle may include a tip formed to penetrate a tissue at the tip end, a groove communicating with the tissue extraction recess, and a needle tube having a tube shape connected to a rear end portion of the inner needle to be communicate with the groove.

The sliding block may include a negative pressure transfer pipe that is formed to communicate the first negative pressure space and the groove.

A first negative pressure valve formed between the first negative pressure space and the negative pressure transfer pipe may be opened to transfer a negative pressure formed in the first negative pressure space to the tissue extraction recess when the sliding block moves backward, and closed to prevent a positive pressure formed in the first negative pressure space from being transferred to the tissue extraction recess when the sliding block moves forward, and a second negative pressure valve formed between the second negative pressure space and an open portion of a rear end of the needle tube, may be closed to prevent a positive pressure formed in the second negative pressure space from being transferred to the tissue extraction recess when the sliding block moves backward, and open to transfer a negative pressure formed in the second negative pressure space to the tissue extraction recess when the sliding block moves forward.

The sliding block may include a mother block disposed to slide along the outer circumference of the inner needle and having a space formed in a lengthwise direction therein, and a baby block, to which the outer needle is fixed, assembled to move backward in the mother block.

The baby block may include a baby block lever to enable assembly and disassembly of the baby block with respect to the mother block.

The mother block and the baby block may be disassembled from each other by manipulating the baby block lever, and an extracted tissue sample may be collected as the tissue extraction recess of the inner needle is opened by pulling the baby block lever backward.

The loading unit may include a first spring, a flange block, and a second spring, which are connected to a rear portion of the sliding block and sequentially arranged in the housing.

The first spring may include a tension spring, and the second spring may be a compression spring.

The first spring of the loading unit may be loaded by pulling the loading handle, and the second spring of the loading unit is loaded by pushing the loading handle.

The loading unit may include the first spring having one side connected to the front portion of the housing and the other side connected to the front portion of the sliding block in the housing, the flange block disposed at the rear portion of the sliding block, and the second spring having one side connected to the flange block and the other side connected to the rear portion of the housing.

The first spring and the second spring may be compression springs, and the elasticity of the second spring may be greater than the elasticity of the first spring.

The second spring may be loaded by pulling the loading handle, and the first spring may be loaded by a compression force generated when the second spring is shot.

The loading unit may further include a first catch device fixing the sliding block, and a second catch device fixing the flange block.

As the first catch device is released, the sliding block may be shot backward by a restoration force of the first spring, and then the sliding block moved backward may release the second catch device and the flange block may push the sliding block by a restoration force of the second spring, thereby shooting the sliding block forward.

After forward shot of the sliding block, the sliding block may return to a fixed position by a restoration force of the second spring and may be automatically fixed to the first catch device.

The first catch device may further include a shoot button releasing the first catch device, a first catch protrusion hinge-coupled to the shoot button and protruding into the housing to fix the first sliding block, and a first restoring spring transferring a restoration force to the shoot button and the first catch protrusion so that fix the shoot button and the first catch protrusion fixes the first sliding block when the first sliding block arrives at a fixing position.

The second catch device may include a second catch protrusion fixing the flange block, and a second restoring spring transferring a restoration force to the second catch protrusion so that the second catch protrusion fixes the flange block when the flange block arrives at the fixing position.

The sliding block may include a spring accommodation portion formed inwardly in a contact surface where the first spring or the second spring contacts the sliding block so as to accommodate the first spring or the second spring, and the flange block may include a spring accommodation portion formed inwardly in a contact surface where the first spring or the second spring contacts the flange block so as to accommodate the first spring or the second spring.

The sliding block may further include at least one O-ring to reduce friction with the sliding space and to keep sealing during sliding in the housing.

The sliding block may include a detachment prevention step formed in a front portion and a rear portion of the sliding block to prevent detachment of the O-ring.

The loading handle may be disposed to surround a rear portion of the housing.

According to another aspect of the present disclosure, a method of operating a disposable cutting biopsy device includes forming the first negative pressure space in the front portion as the sliding block moves backward, and starting accommodation of a specimen tissue in the tissue extraction recess opened by a backward movement of the outer needle; applying a negative pressure formed in the first negative pressure space to the tissue extraction recess, completing the accommodation of the specimen tissue in the tissue extraction recess, starting cutting of the specimen tissue by the outer needle as the sliding block is shot forward and the second negative pressure space is formed in a rear portion of the housing, applying a negative pressure formed in the second negative pressure space to the tissue extraction recess, and completing cutting of the specimen tissue with the outer needle.

A structure of a needle of a cutting biopsy device according to another first embodiment of the present disclosure may include an inner needle having an assigned length in the back and forth direction, the inner needle including a head part having a leading tip formed thereon to penetrate and enter at least a biological tissue, and a body part extending in a lengthwise direction from the head part and having a tissue extraction recess for accommodating a biological tissue formed to be concave therein, and an outer needle having a needle tube including the inner needle therein, and moving to accommodate the biological tissue in the tissue extraction recess with a seal by a cutting blade formed at one side end that is the same as the leading tip side according to a relative motion with the inner needle in the lengthwise direction, by manipulation of an operator, wherein when the front cross-section of the leading tip may be divided into four quadrants, in which the upper right quadrant is set to a first quadrant, and the other quadrants counterclockwise from the first quadrant may be set to second to fourth quadrants, in a region corresponding to the first and second quadrants, a first inclined surface is formed face one surface in the lengthwise direction including a tip point that is one point in the axis direction of the inner needle, in a region corresponding to the third quadrant, a second inclined surface is formed to face the tip point, and in a region corresponding to the fourth quadrant, a third inclined surface is formed to face the tip point, wherein a point where the first inclined surface, the second inclined surface, and the third inclined surface simultaneously meet is the tip point, wherein engraved pattern is formed on the first inclined surface, the second inclined surface, and the third inclined surface.

In the present embodiment, the first inclined surface is a surface in the lengthwise direction including a tip point that is one point in the axis direction of the inner needle parallel to the axis of the inner needle and orthogonal to a direction toward a tip point of the inner needle, and may be inclined to the one surface and may meet the one surface at the leading tip.

In the present embodiment, the second inclined surface and the third inclined surface may be symmetric to the left and right with the tip point as a center.

In the present embodiment, the length of the first inclined surface in the lengthwise direction may be greater than the lengths of the second inclined surface and the third inclined surface in the lengthwise direction.

A structure of a needle of the cutting biopsy device according to another first embodiment of the present disclosure may include an inner needle having an assigned length in the back and forth direction, the inner needle including a head part having a leading tip formed thereon to penetrate and enter at least a biological tissue and an extension portion extending from the leading tip, and a body part extending in a lengthwise direction from the head part and having a tissue extraction recess for accommodating a biological tissue formed to be concave therein, and an outer needle having a needle tube including the inner needle therein, and moving to accommodate the biological tissue in the tissue extraction recess with a seal by a cutting blade formed at one side end that is the same as the leading tip side according to a relative motion with the inner needle in the lengthwise direction, by manipulation of an operator, wherein the outer diameter of the outer needle is the same as the maximum outer diameter of the head part of the inner needle.

In the present embodiment, the maximum outer diameter of the body part is less than the maximum outer diameter of the head part, and the maximum advancement position of the cutting blade of the outer needle is behind the leading tip based on a direction toward the leading tip of the inner needle compared with a region where at least leading tip of the head part of the inner needle is formed. A structure of a needle of the cutting biopsy device according a second embodiment of the present disclosure may include an inner needle having an assigned length in the back and forth direction, the inner needle including a head part having a leading tip formed thereon to penetrate and enter at least a biological tissue, and a body part extending in a lengthwise direction from the head part and having a tissue extraction recess for accommodating a biological tissue formed to be concave on a lower surface of a region adjacent to an area where the leading tip is formed, and an outer needle having a needle tube including the inner needle therein, and moving to accommodate the biological tissue in the tissue extraction recess with a seal by a cutting blade formed at one side end that is the same as the leading tip side according to a relative motion with the inner needle in the lengthwise direction, by manipulation of an operator, wherein the region adjacent to the leading tip of the outer needle is formed to have the same inclination degree as the leading tip of the inner needle in the form in which a surface formed by the leading tip of the inner needle extends.

In the present embodiment, the inner needle may further include a negative pressure portion formed in a region at one side in a direction opposite to the direction toward the leading tip in the region adjacent to the area where the tissue extraction recess is formed, to be concave to allow air pass through the tissue extraction recess and a hole, thereby lowering air pressure of the tissue extraction recess.

In the present embodiment, the inner needle may further include a negative pressure portion formed in a region at one side in a direction opposite to the direction toward the leading tip in the region adjacent to the area where the tissue extraction recess is formed, to be connected to the tissue extraction recess in the form of a groove, thereby lowering air pressure of the tissue extraction recess.

In the present embodiment, an engraved pattern may be formed on at least an inclined surface formed by the leading tips of the outer needle and the inner needle and in one region adjacent to the leading tip of an outer surface of the outer needle.

### BRIEF DESCRIPTION OF DRAWINGS

FIG. 1 is a conceptual diagram of a cutting biopsy device of the related art.

FIG. 2 is a perspective view of a disposable cutting biopsy device according to an embodiment of the present disclosure.

FIG. 3 is a cross-sectional view of a disposable cutting biopsy device according to an embodiment.

FIG. 4 is a side view of a needle set according to an embodiment.

FIG. 5 is a side view of an inner needle according to an embodiment.

FIG. 6 is a perspective view of an inner needle of a needle set according to an embodiment.

FIG. 7 is a side view illustrating a combination of the sliding block and the needle set according to an embodiment.

FIGS. 8A, 8B, and 8C are side views of a sliding block according to an embodiment.

FIG. 9 is a side view illustrating a combination of the sliding block and the needle set according to another embodiment.

FIG. 10 is a side view of a housing according to an embodiment.

FIGS. 11A and 11B are conceptual diagrams of generation of a negative pressure according to a reciprocating motion of a sliding block according to an embodiment.

FIGS. 12A and 12B illustrate a first negative pressure valve and a second negative pressure valve according to an embodiment.

FIG. 13 is a flowchart of a negative pressure cutting operation method according to a reciprocating motion of a sliding block according to an embodiment.

FIG. 14 is a perspective view illustrating a combination of the needle set, the sliding block, and the loading unit according to an embodiment.

FIG. 15 is a cross-sectional view illustrating a combination of the loading unit, the loading handle, and the sliding block of the housing according to an embodiment.

FIG. 16 is a perspective view of the loading handle according to an embodiment.

FIG. 17 is a disassembled view of a hook device according to an embodiment.

FIG. 18 is a conceptual view illustrating a loading operation of a disposable cutting biopsy device according to an embodiment.

FIG. 19 illustrates a sequence of loading and operation of a cutting biopsy device according to an embodiment.

FIG. 20 illustrates a state of a leading end portion of a needle set according to an embodiment.

FIG. 21 is a cross-sectional view illustrating a combination of a housing, a loading unit, and a sliding block according to another embodiment.

FIG. 22 is a side view of a sliding block according to another embodiment sliding block.

FIG. 23 is a side cross-sectional view of a structure of a needle of the cutting biopsy device according to the related art.

FIG. 24 is a side view of a structure of a needle of a cutting biopsy device according to a first embodiment of the present disclosure.

FIGS. 25A, 25B, and 25C are respectively a plan view, a side view, and a bottom view for describing a modified example of a structure of a needle of a cutting biopsy device according to a first embodiment of the present disclosure.

FIGS. 26A and 26B are perspective views for describing a structure of an inner needle of a cutting biopsy device according to a first embodiment of the present disclosure.

FIGS. 27 and 28 are front views for describing a structure of a leading tip of a cutting biopsy device according to a first embodiment of the present disclosure.

FIG. 29 is a side cross-sectional view of a structure of a needle of a cutting biopsy device according a second embodiment of the present disclosure.

FIGS. 30 and 31 are side cross-sectional views of a structure of a needle of a cutting biopsy device according to a modified example of a second embodiment of the present disclosure.

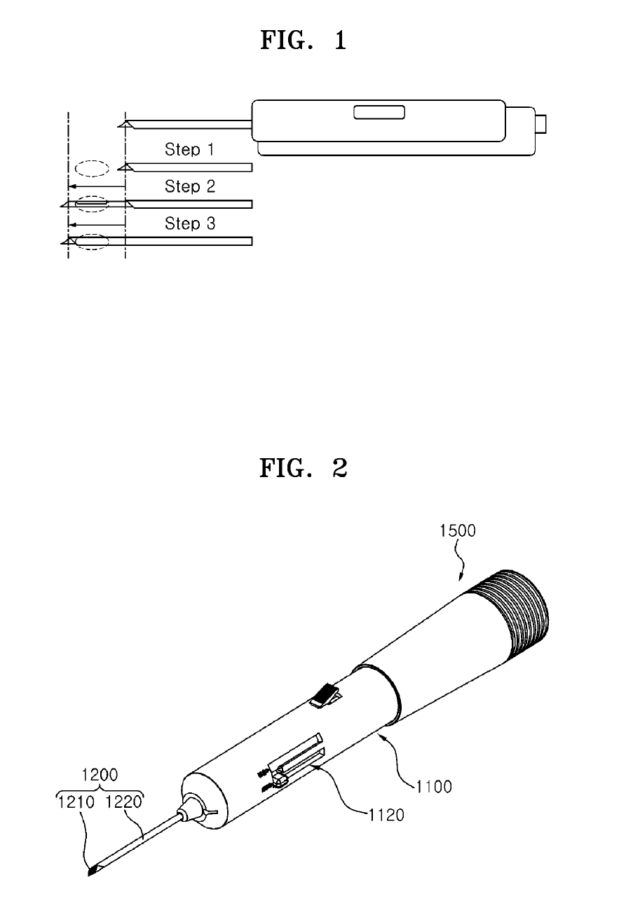

### MODE OF DISCLOSURE

As the disclosure allows for various changes and numerous embodiments,

particular embodiments will be illustrated in the drawings and described in detail in the written description. However, this is not intended to limit the present disclosure to particular modes of practice, and it is to be appreciated that all changes, equivalents, and substitutes that do not depart from the spirit and technical scope of the present disclosure are encompassed in the present disclosure. Throughout the drawings, like reference numerals denote like elements. In the accompanying drawings, the dimensions of structures are exaggerated for clarity of the present disclosure.

Terms such as “first” and “second” are used herein merely to describe a variety of constituent elements, but the constituent elements are not limited by the terms. Such terms are used only for the purpose of distinguishing one constituent element from another constituent element. For example, without departing from the right scope of the present disclosure, a first constituent element may be referred to as a second constituent element, and vice versa.

Terms used in the present specification are used for explaining a specific embodiment, not for limiting the present disclosure. Thus, an expression used in a singular form in the present specification also includes the expression in its plural form unless clearly specified otherwise in context. Also, terms such as “include” or “comprise” may be construed to denote a certain characteristic, number, step, operation, constituent element, or a combination thereof, but may not be construed to exclude the existence of or a possibility of addition of one or more other characteristics, numbers, steps, operations, constituent elements, or combinations thereof. Furthermore, when A “connects” or is “connected” to B, A contacts or is connected to B directly or through at least one constituent element C provided between A and B.

Unless defined otherwise, all terms used herein including technical or scientific terms have the same meanings as those generally understood by those of ordinary skill in the art to which the present disclosure may pertain. The terms as those defined in generally used dictionaries are construed to have meanings matching that in the context of related technology and, unless clearly defined otherwise, are not construed to be ideally or excessively formal. Furthermore, in the claims regarding a method invention, the order of the steps of a method described herein can be changed unless otherwise indicated herein or otherwise clearly contradicted by context.

Hereinafter, the present disclosure will be described in detail by explaining embodiments of the disclosure with reference to the drawings.

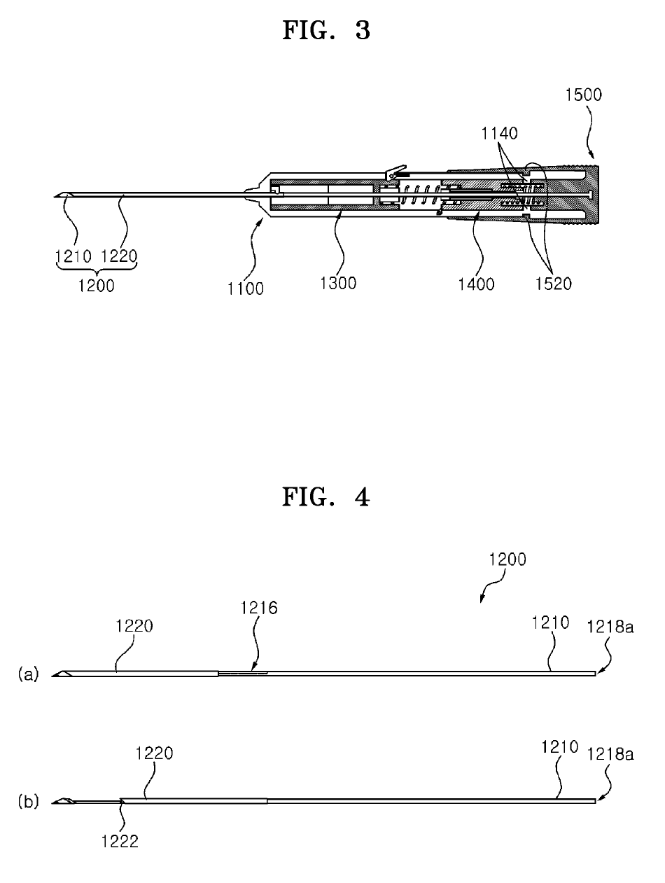

FIG. 2 is a perspective view of a disposable cutting biopsy device according to an embodiment of the present disclosure. FIG. 3 is a cross-sectional view of a disposable cutting biopsy device according to an embodiment.

Referring to FIGS. 2 and 3, a disposable cutting biopsy device according to the present embodiment extracts a tissue through a forward shot after a backward shot of a needle set.

To implement the above, a disposable cutting biopsy device 1000 according to the present embodiment may include a housing 1100 having a sliding space therein, a needle set 1200 disposed in the housing 1100 and extracting a tissue sample by using an outer needle 1220 and an inner needle 1210, a sliding block 1300 connected to the outer needle 1220 and capable of reciprocating back and forth in the sliding space of the housing 1100, a loading unit 1400 shooting the sliding block 1300, and a loading handle 1500 arranged to be connected to the loading unit 1400.

The housing 1100 may have one tubular shape. The housing 1100, as illustrated in FIGS. 2 and 3, may have the sliding space in which the sliding block 1300 and the loading unit 1400 are inserted and freely slide therein.

Accordingly, the sliding block 1300 and the loading unit 1400 may slide in the housing 1100 without adding another complicated assembly process and structure.

Furthermore, the housing 1100 may have a shape of an open rear portion. Accordingly, the rear portion of the housing 1100 may be covered by, for example, the loading handle 1500, and connected to the loading unit 1400 provided in the housing 1100.

The housing 1100 may include a coupling guide at the rear portion so that the loading handle 1500 is coupled to the rear portion of the housing 1100 and thus to be fixed after a loading operation.

The loading unit, the loading handle, and the coupling guide are described below in detail with reference to the drawings.

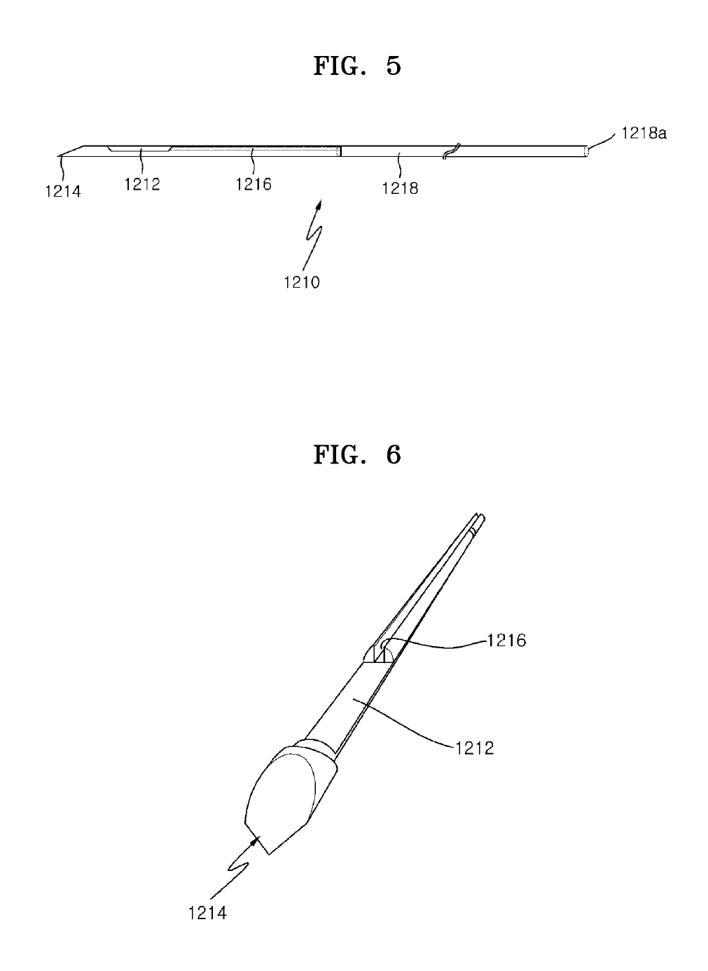

FIG. 4 is a side view of a needle set according to an embodiment. FIG. 5 is a side view of an inner needle according to an embodiment. FIG. 6 is a perspective view of an inner needle of a needle set according to an embodiment.

Referring to FIG. 4, the needle set 1200 is disposed in the housing 1100 by penetrating through a front portion hole of the housing 1100.

The needle set 1200 may include the inner needle 1210 having a tissue extraction recess 1212, and the outer needle 1220 having the inner needle 1210 therein and reciprocating along an outer circumference of the inner needle 1210 to cut a tissue accommodated in the tissue extraction recess 1212.

A cutting blade 1222 is formed on a leading end portion of the outer needle 1220 and reciprocates or glides along the outer circumference of the inner needle 1210 to cut the tissue accommodated in the tissue extraction recess 1212.

The inner needle 1210 may include a leading tip 1214 at a leading end of the inner needle 1210 to penetrate a tissue, and the tissue extraction recess 1212 in the rear of the leading tip 1214.

Furthermore, as illustrated in FIGS. 5 and 6, the inner needle 1210 may include a groove 1216 that communicates with the tissue extraction recess 1212 and a needle tube 1218 having a tubular shape connected to a rear end portion of the inner needle 1210 to communicate with the groove 1216.

The groove 1216 and the needle tube 1218 may transfer a negative pressure generated in a negative pressure space that is instantly formed by a reciprocating motion of the sliding block 1300 in the housing 1100 to the tissue extraction recess 1212, and thus a specimen tissue that is introduced into the tissue extraction recess 1212 opened by a backward movement of the outer needle 1220 is further smoothly absorbed, and during cutting by a forward shot of the outer needle 1220, the introduced specimen tissue may be fixed to the tissue extraction recess 1212, thereby facilitating a cutting process.

The outer needle 1220 that cuts the specimen tissue by using the cutting blade 1222 while reciprocating or gliding along the outer circumference of the inner needle 1210 may be fixed to the sliding block 1300 disposed in the housing 1100 to reciprocate and glide along the outer circumference of the inner needle 1210. The outer needle 1220, when the sliding block 1300 and the loading unit 1400 are loaded by the loading handle 1500, may arrive with the inner needle 1210 at a tissue target to be extracted and may be inserted thereinto, and may cut the tissues accommodated in the tissue extraction recess 1212 by a forward shot after a backward shot of the sliding block 1300 by a shooting operation of the loading unit 1400.

The loading and shot of the sliding block and the outer needle and a negative pressure structure are described below in detail with reference to the drawings.

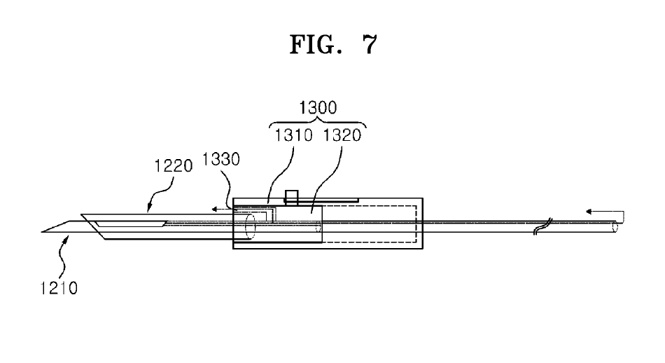

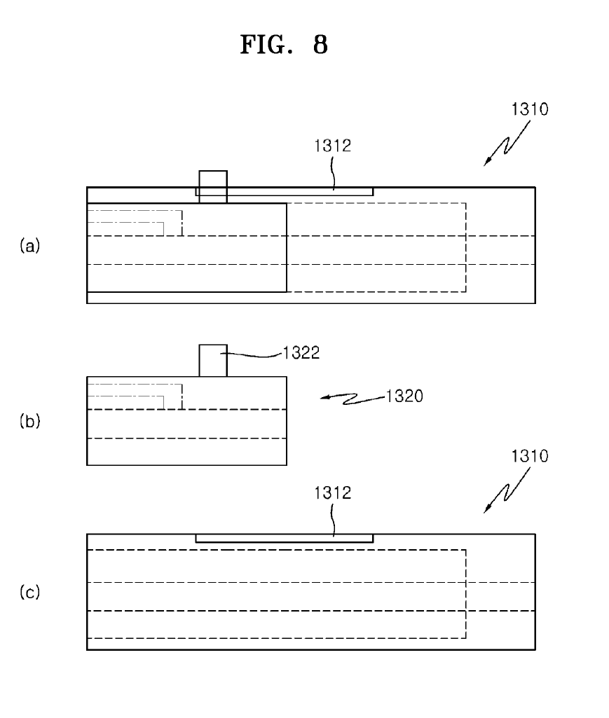

FIG. 7 is a side view illustrating a combination of the sliding block and the needle set according to an embodiment. FIGS. 8A, 8B, and 8C are side views of a sliding block according to an embodiment. FIG. 9 is a side view illustrating a combination of the sliding block and the needle set according to another embodiment.

The sliding block 1300, as illustrated in FIGS. 7 and 8, may be connected to a rear end portion of the outer needle 1220 and reciprocate or glide along the outer circumference of the inner needle 1210.

The sliding block 1300 may include a mother block 1310, a baby block 1320 and a negative pressure transfer pipe 1330 that communicates with the groove 1216 of the inner needle 1210.

FIG. 8A is a side view illustrating that the mother block and the baby block are assembled with each other. FIG. 8B is a side view of the baby block. FIG. 8C is a side view of the mother block.

Referring to FIGS. 8A to 8C, the mother block 1310 may be disposed to slide along the outer circumference of the inner needle 1210, and may have a space in a lengthwise direction therein. The outer needle 1220 may be fixed to a leading end portion of the baby block 1320, and the baby block 1320 may be coupled to the mother block 1310 to move back and forth in the mother block 1310.

Furthermore, to enable the assembly and disassembly of the mother block 1310 and the baby block 1320, the baby block 1320 may include a baby block lever 1322, and the mother block 1310 may include a baby block lever guide 1312 to assist the operation of the baby block lever 1322.

As illustrated in FIGS. 7 and 8, an inner space of the mother block 1310 may be formed to be longer than the length of the baby block 1320, and during a negative pressure cutting operation, the mother block 1310 and the baby block 1320 are operated in an assembled state, and during extraction of a specimen, the baby block 1320 may be disassembled from the mother block 1310 and move backward by manipulating the baby block lever 1322.

A distance that the baby block 1320 moves backward may be a distance that the outer needle 1220 moves backward simultaneously with the backside movement of the baby block 1320 to expose the tissue extraction recess 1212, and accordingly, the length of the inner space of the mother block 1310 and the length of the baby block 1320 may be determined.

The baby block lever guide 1312 may have a “ [symbol] ” shape for the assembly and disassembly of the mother block 1310 and the baby block 1320. For example, as the baby block lever 1322 moves backward along a linear guide of the baby block lever 1322 that is in a disassembly mode of the baby block lever guide 1312, the baby block 1320 is disassembled from the mother block 1310 and may move backward with the outer needle 1220 without the backward movement of the mother block 1310. Accordingly, the tissue extraction recess 1212 may be exposed without performing a loading operation of the loading handle 1500 and the loading unit 1400.

Furthermore, when the baby block lever 1322 is moved to a rotation guide of the baby block lever 1322 that is in an assembly mode of the baby block lever guide 1312, the baby block 1320 is assembled with the mother block 1310 and thus backward movement of the sliding block 1300 is possible by the loading operation of the loading handle 1500 and the loading unit 1400.

Although the shape of the baby block lever guide 1312 according to one embodiment of the present disclosure is described to be “ [symbol] ”, the present disclosure is not limited thereto, and a mode guide 1120 may be formed in a linear shape. Accordingly, the baby block 1320 and the mother block 1310 may be assembled and disassembled by pressing a button.

The negative pressure transfer pipe 1330 may allow a first negative pressure space and the groove 1216 to communicate with or be cut off from each other, the first negative pressure space being instantly formed in a front portion of the housing 1100 as the sliding block 1300 reciprocates or slides in the sliding space in the housing 1100.

Furthermore, the negative pressure transfer pipe 1330 may be formed in the baby block 1320, as illustrated in FIG. 8, but the negative pressure transfer pipe 1330 may be formed in the mother block 1310, not in the baby block 1320. Furthermore, as illustrated in FIG. 9, the negative pressure transfer pipe 1330 may be formed throughout the baby block 1320 and the mother block 1310. The thickness or diameter of each of the mother block 1310 and the baby block 1320 of the present disclosure may be reduced sufficiently to reduce the thickness of the disposable cutting biopsy device. Considering the assembly relation of or the formation of a space or a gap between the mother block 1310 and the baby block 1320, the negative pressure transfer pipe 1330 may be optionally formed in the mother block 1310 or throughout the mother block 1310 and the baby block 1320.

To enable the manipulation of the baby block lever 1322 after the sliding block 1300 and the needle set 1200 are disposed in the housing 1100, the sliding block 1300 and the needle set 1200 are disposed by being assembled with the housing 1100.

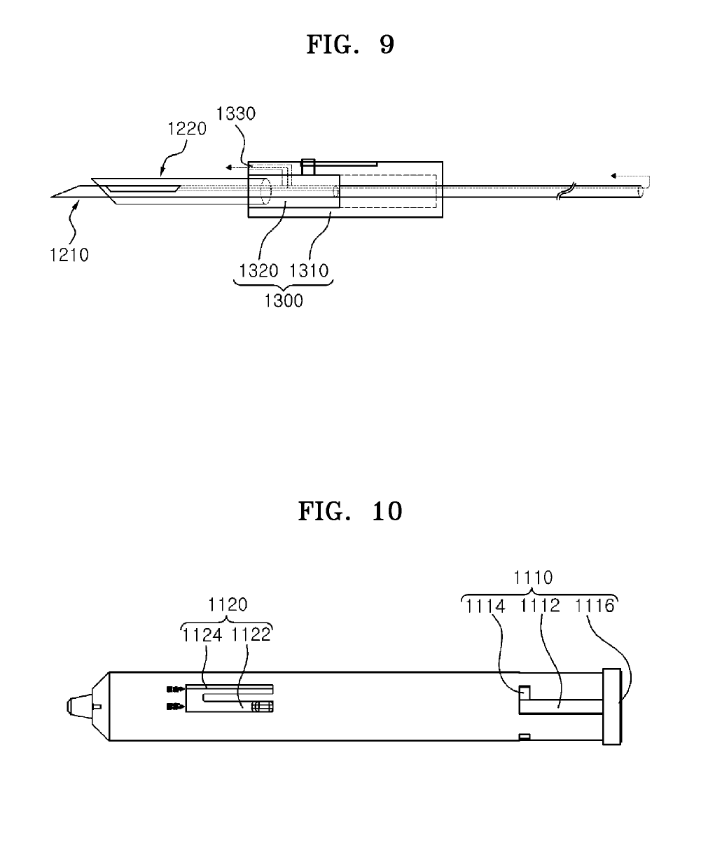

FIG. 10 is a side view of a housing according to an embodiment.

As illustrated in FIG. 10, the housing 1100 may include the mode guide 1120 on a side surface of the housing 1100.

The mode guide 1120 may include an operation mode guide 1122 in which the baby block 1320 is moved by being assembled with the mother block 1310 and a safe mode guide 1124 in which the baby block 1320 moves separately from the mother block 1310.

In other words, when a user locates the baby block lever 1322 in the operation mode guide 1122, the baby block lever 1322 is placed in the rotation guide, and the baby block 1320 is assembled with the mother block 1310 so that the sliding block 1300 is to be loaded and shot based on the loading operation of the loading unit 1400 and the loading handle 1500, thereby starting the negative pressure cutting operation.

Furthermore, when the user locates the baby block lever 1322 in the safe mode guide 1124, the baby block lever 1322 is placed in the linear guide, and the baby block 1320 and the mother block 1310 are disassembled from each other, and thus the baby block 1320 solely moves backward with the outer needle 1220.

Although as one embodiment of the present disclosure, the mode guide 1120 has a “⊏” shape including the operation mode guide 1122 and the safe mode guide 1124, the present disclosure is not limited thereto, and the mode guide 1120 may have a linear shape, and thus the baby block 1320 and the mother block 1310 may be switched to the safe mode by pressing a button.

When the mother block 1310 and the baby block 1320 are assembled with each other, the sliding block 1300 and the outer needle 1220 may perform the negative pressure cutting operation based on the operation of the loading unit 1400 and the loading handle 1500.

Furthermore, the housing 1100 may include one or more coupling guides 1110 to facilitate a movement of the loading handle 1500 and to load the loading unit 1400, the sliding block 1300, and the outer needle 1220.

Detailed descriptions about the loading handle 1500 and the coupling guide 1110 are presented below in detail with reference to drawings.

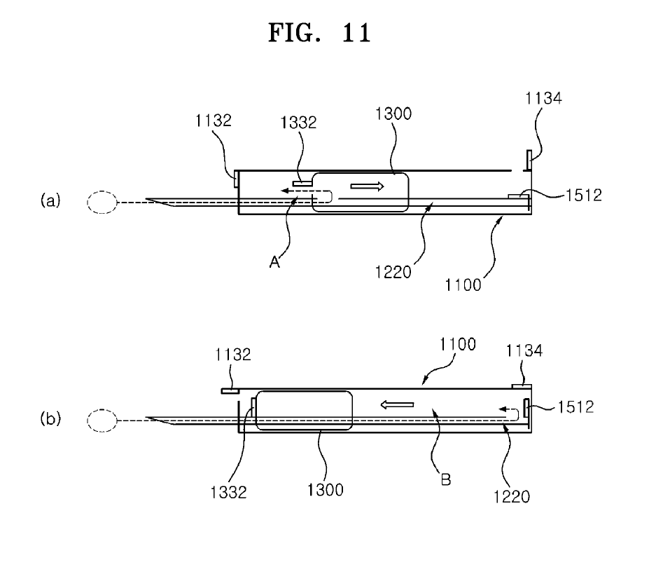

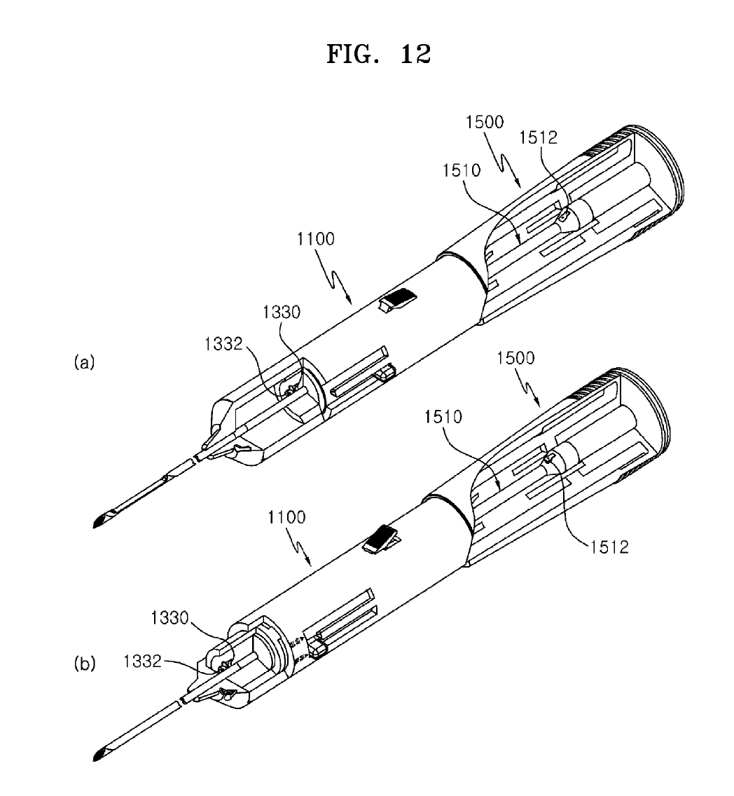

FIGS. 11A and 11B are conceptual diagrams? of generation of a negative pressure according to a reciprocating motion of a sliding block according to an embodiment. FIGS. 12A and 12B illustrate a first negative pressure valve and a second negative pressure valve according to an embodiment.

FIG. 11A is a conceptual diagram illustrating that the sliding block 1300 moves backward in the sliding space of the housing 1100, and FIG. 11 B is a conceptual diagram illustrating that the sliding block 1300 moves forward in the sliding space of the housing 1100.

Referring to FIGS. 11A and 11B, as the sliding block 1300 moves in the sliding space of the housing 1100, the volume of space of the front portion and the rear portion of the housing 1100 may vary. Accordingly, when the sliding block 1300 moves backward in the sliding space, a first negative pressure space A is formed in the front portion of the housing 1100, and when the sliding block 1300 moves forward, a second negative pressure space B is formed in the rear portion of the housing 1100.

As the first negative pressure space A and the second negative pressure space B are instantly formed due to a rapid reciprocating motion of the sliding, a positive pressure and a negative pressure may be generated in the first negative pressure space A and the second negative pressure space B.

In other words, when the sliding block 1300 moves backward in the sliding space of the housing 1100, the first negative pressure space A is formed and simultaneously a negative pressure is generated, and thus the volume of the second negative pressure space B decreases and a positive pressure is generated. In contrast, when the sliding block 1300 moves forward in the sliding space of the housing 1100, the volume of the first negative pressure space A decreases and a positive pressure is generated, and thus the second negative pressure space B is formed and simultaneously a negative pressure is generated.

The formed negative pressure and positive pressure may be transferred to the tissue extraction recess 1212 or may be cut off by a plurality of valves formed on the sliding block 1300, the housing 1100, and the inner needle 1210, as illustrated in FIG. 9.

The housing 1100 may further include a plurality of positive pressure valves 1132 and 1134. As the sliding block 1300 moves forward, to remove the positive pressure formed in the first negative pressure space A, a first positive pressure valve 1132 may be further included in the front portion of the housing 1100 and, as the sliding block 1300 moves backward, to remove the positive pressure formed in the second negative pressure space B, a second positive pressure valve 1134 may be further included in the rear portion of the housing 1100.

Furthermore, in one embodiment of the present disclosure, to transfer the negative pressure, not the positive pressure formed in the first negative pressure space A and the second negative pressure space B, to the tissue extraction recess 1212, a first negative pressure valve 1332 formed between the first negative pressure space A and the negative pressure transfer pipe 1330 and a second negative pressure valve 1512 between the second negative pressure space B and an open portion 1218a at a rear end of the needle tube 1218 may be further included. In this state, as the rear end portion of the needle tube 1218 may be fixed to the loading handle 1500, the second negative pressure valve 1512 may be formed between the loading handle 1500 and the second negative pressure space B.

Although the second negative pressure valve 1512 may be formed at the rear end of the needle tube 1218, according to another embodiment, as illustrated in FIG. 12, the second negative pressure valve 1512 may be formed at a loading rod of the loading handle.

In other words, as illustrated in FIG. 11A), when the sliding block 1300 moves backward, the first positive pressure valve 1132 is closed so that a negative pressure may be generated in the first negative pressure space A and the first negative pressure valve 1332 may be open so that the generated negative pressure is transferred to the tissue extraction recess 1212. To prevent the positive pressure generated in the second negative pressure space B from being transferred to the tissue extraction recess 1212, the second negative pressure valve 1512 may be closed and the second positive pressure valve 1134 may be opened.

Furthermore, when the sliding block 1300 moves forward, the first positive pressure valve 1132 may be open to prevent the negative pressure formed in the first negative pressure space A from being transferred to the tissue extraction recess 1212, and the first negative pressure valve 1332 may be closed. To transfer the negative pressure formed in the second negative pressure space B to the tissue extraction recess 1212, the second negative pressure valve 1512 may be open and the second positive pressure valve 1134 may be closed.

Accordingly, a reciprocating motion of the sliding block 1300 in the sliding space of the housing 1100 may cause cutting of a specimen tissue accommodated in the tissue extraction recess 1212 due to the movement of the outer needle 1220 and the negative pressure applied to the tissue extraction recess 1212.

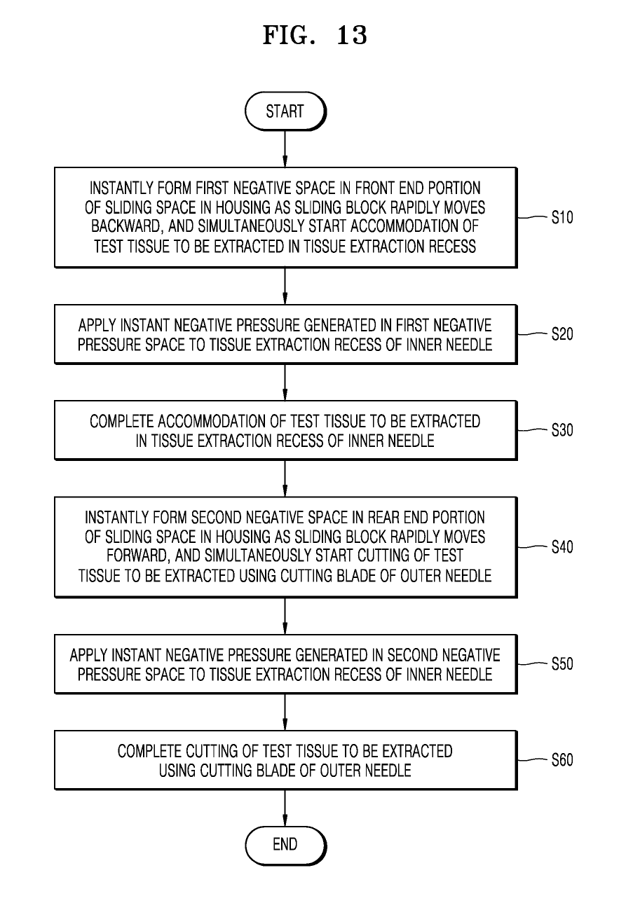

FIG. 13 is a flowchart of a negative pressure cutting operation method according to a reciprocating motion of a sliding block according to an embodiment.

Referring to FIGS. 11A and 11B and 13, when the needle set 1200 is injected into a specimen tissue that the user desires to extract, the user starts to rapidly move the sliding block 1300 and the outer needle 1220 backward. In this state, the first negative pressure space A is instantly formed in the front portion of the sliding space of the housing 1100, and simultaneously the tissue extraction recess 1212 is opened and the specimen tissue is accommodated in the tissue extraction recess 1212 (S10).

A negative pressure is generated in the first negative pressure space A formed in S10, the generated negative pressure is rapidly transferred to the tissue extraction recess 1212 via the negative pressure transfer pipe 1330 of the sliding block 1300 and the groove 1216 (S20). In this state, to prevent the positive pressure generated in the second negative pressure space B from being transferred to the tissue extraction recess 1212, an open portion of a rear end of the inner needle 1210 is closed.

The negative pressure transferred to the tissue extraction recess 1212 further absorbs the specimen tissue accommodated in the tissue extraction recess 1212 by a tissue pressure, thereby completing the accommodation (S30).

The sliding block and the outer needle 1220 having completed the backward movement start to move forward rapidly. In this state, the first negative pressure space A formed in the front portion of the sliding space of the housing 1100 disappears and a positive pressure is generated, and the second negative pressure space B is formed in the rear portion of the sliding space of the housing 1100, and the cutting blade 1222 of the outer needle 1220 starts to cut the specimen tissue (S40).

A negative pressure is formed in the second negative pressure space B formed in S40, the generated negative pressure is rapidly transferred to the tissue extraction recess 1212 via the needle tube 1218 (S50), thereby making the accommodation of the specimen tissue being cut further firm. In this state, to prevent the positive pressure generated in the first negative pressure space A from being transferred to the tissue extraction recess 1212, the negative pressure transfer pipe 1330 and the first negative pressure space A do not communicate with each other.

As the forward movement of the sliding block 1300 and the outer needle 1220 is completed, the cutting of the specimen tissue that the user desires is completed (S60) and the negative pressure cutting operation is completed.

When the negative pressure cutting operation is completed, the user may disassemble the mother block 1310 and the baby block 1320 of the sliding block 1300 from each other to expose the tissue extraction recess 1212, thereby extracting the cut specimen tissue.

The sliding block 1300 and the outer needle 1220 may be loaded according to the movement of the loading unit 1400 and the loading handle 1500 and may generate a negative pressure.

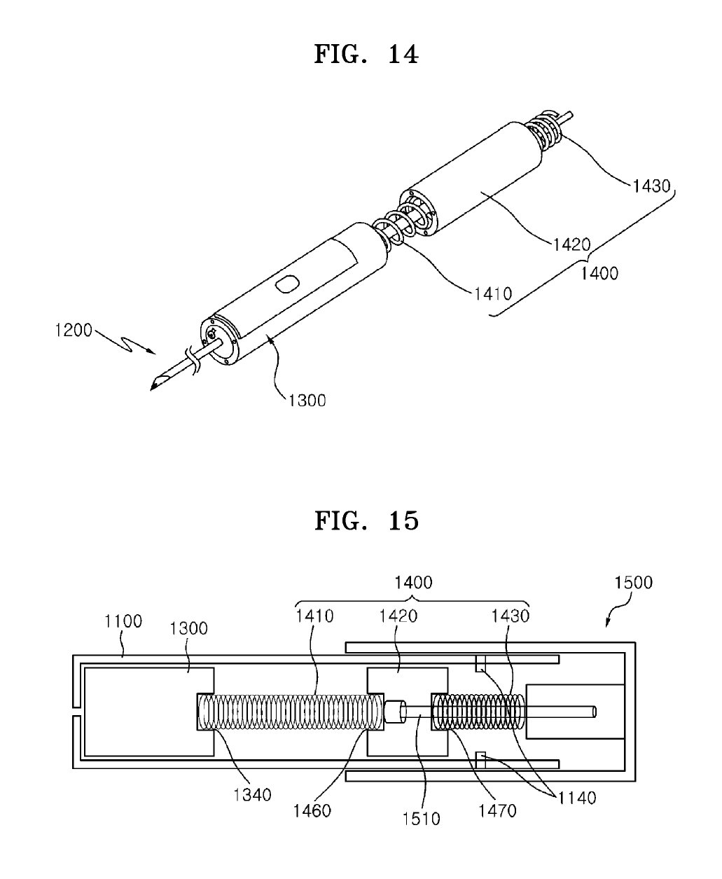

FIG. 14 is a perspective view illustrating a combination of the needle set, the sliding block, and the loading unit according to an embodiment. FIG. 15 is a cross-sectional view illustrating a combination of the loading unit, the loading handle, and the sliding block of the housing according to an embodiment.

As illustrated in FIGS. 14 and 15, the loading unit 1400 is connected to the sliding block 1300 and disposed in the sliding space of the housing 1100.

For example, the loading unit 1400 may include a first spring 1410, a flange block 1420, and a second spring 1430, which are connected to a rear portion of the sliding block 1300 and sequentially arranged in the sliding space of the housing 1100.

In detail, the sliding block 1300 and the flange block 1420 are connected by the first spring 1410. Furthermore, the second spring 1430 is connected to the flange block 1420 and is located between the flange block 1420 and the loading handle 1500.

The inner needle 1210 may penetrate the sliding block 1300, the first spring 1410, the flange block 1420, and the second spring 1430, which may be provided to slide along the outer circumference of the inner needle 1210.

In this state, the first spring 1410 that is a tension spring my pull the sliding block 1300 and the flange block 1420 close to each other, and the second spring 1430 that is a compression spring may push the flange block 1420 and the loading handle 1500 away from each other.

The sliding block 1300 and the flange block 1420 may be disposed in the housing 1100, may smoothly slide along the outer circumference of the inner needle 1210, and may be provided close to the housing 1100 without a gap therebetween. For example, the sliding block 1300 and the flange block 1420 may be provided close to the housing 1100 without a gap therebetween to prevent air exchange therebetween so that, as the sliding block 1300 slides back and forth, a negative pressure or a positive pressure may generated in the first negative pressure space A formed in front of the sliding block 1300 and the second negative pressure space B formed in the rear of the sliding block 1300.

The loading unit 1400 may be connected to the loading handle 1500, as illustrated in FIG. 15, and may perform a loading operation.

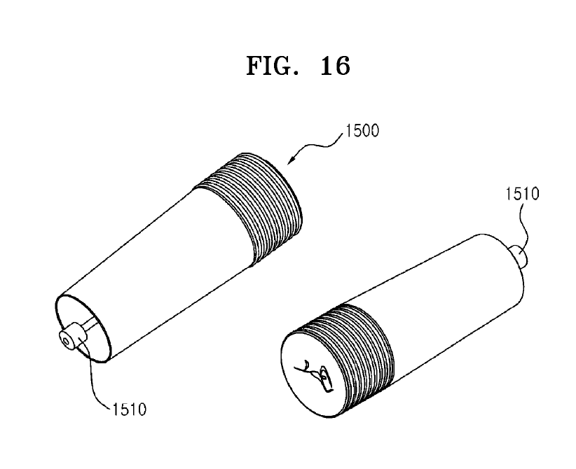

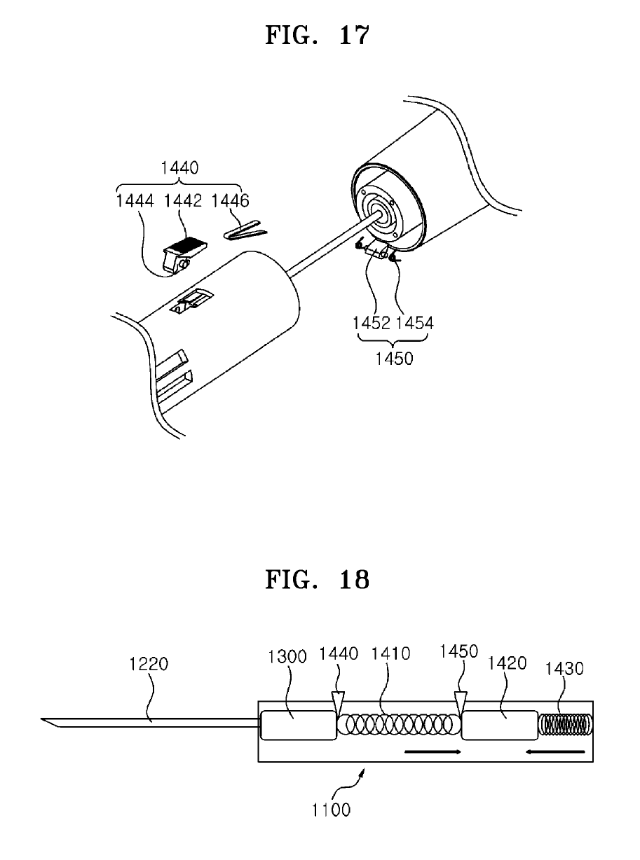

FIG. 16 is a perspective view of the loading handle according to an embodiment. FIG. 17 is a disassembled view of a hook device according to an embodiment. FIG. 18 is a conceptual view illustrating a loading operation of a disposable cutting biopsy device according to an embodiment.

The loading handle 1500 may be connected to the flange block 1420 to load the loading unit 1400.

The loading handle 1500, as illustrated in FIGS. 2, 3, 15, and 16, may have a shape surrounding the rear portion of the housing 1100. In particular, the loading handle 1500 may include a loading rod 1510 connected to the flange block 1420 so that the flange block 1420 may move backward according to the backward movement of the loading handle 1500. The loading rod 1510 may move backward because the flange block 1420 is caught by the loading rod 1510 when the loading handle 1500 moves backward. The loading rod 1510 may be formed to penetrate the flange block 1420 in a lengthwise direction of the housing 1100, as illustrated in FIG. 15, and may have a shape of a hook or a connecting rod, not a rod, but may not be limited thereto.

To facilitate the loading operation, the loading handle 1500 may be fixed after moving along the coupling guide 1110 formed in the housing 1100. The loading handle 1500 may include a coupling protrusion to move along the coupling guide 1110 formed in the housing 1100 and to be fixed thereto.

In this state, as illustrated in FIG. 15, to prevent the flange block 1420 from being detached from the housing 1100, a flange block support portion 1140 may be formed toward the inside of the housing 1100.

In detail, when the user pulls the loading handle 1500 backward, the flange block 1420 moves backward, and thus the flange block 1420 is prevented from being detached to the outside of the housing, thereby easily loading the flange block 1420 and the second spring 1430.

The coupling guide 1110 may be formed in the rear portion of the housing 1100, as illustrated in FIGS. 3 and 10, to be coupled with the loading handle 1500. The coupling guide 1110 may include a linear guide 1112 to smoothly guide a motion of the loading handle 1500 without distortion even when the user pulls or pushes the loading handle 1500. Furthermore, the coupling guide 1110 may include a rotation guide 1114 to compress the first spring 1410 through the linear guide 1112 and then rotate and fix the loading handle 1500 to prevent the loading from being removed by a compression force. For example, the coupling guide 1110 may have a “ [symbol] ” shape. Accordingly, the loading handle 1500 may be guided along the coupling guide 1110 by the coupling protrusion 1520. Furthermore, the coupling guide 1110 may include a detachment prevention step 1116 at an opposite end of the rotation guide 1114 so that the loading handle 1500 may not be detached from the housing 1100 and the coupling guide 1110.

The loading unit 1400 and the sliding block 1300 are loaded by a first catch device 1440 and a second catch device 1450 formed in the loading handle 1500 and the housing 1100 to be shot in the housing 1100 through a restoration force of the first spring 1410 and the first spring 1410, and accordingly, the outer needle 1220 is also loaded.

As illustrated in FIG. 18, before shooting, the sliding block 1300 and the flange block 1420 are loaded by being fixed by the first spring 1410 and the first catch device 1440 and the second catch device 1450 not to be operated by a force transferred from the first spring 1410, and the loading unit 1400, the sliding block 1300, and the outer needle 1220 are shot by releasing the first catch device 1440 and the second catch device 1450.

Referring to FIG. 17, the first catch device 1440 may be disposed at a region of a side surface of the housing 1100, may fix the sliding block 1300 at a first loading position, and by removing the fixing, may shoot the loading unit 1400 and the outer needle 1220 backward toward the flange block 1420 by a tensile force of the first spring 1410.

The first catch device 1440 may include a shoot button 1442, and the user may start a shooting operation of the outer needle 1220 by removing the fixing of the sliding block 1300 simply by pressing the shoot button 1442.

Furthermore, the first catch device 1440 may include a first catch protrusion 1444 that is hinge-coupled to the shoot button 1442 and protrudes inward to fix the sliding block 1300.

The first catch protrusion 1444 may have the sliding block 1300 fixed at a loading position, and the sliding block 1300 may have a first catch point at any one side surface corresponding to the first catch protrusion 1444 so that the first catch protrusion 1444 may firmly fix the sliding block 1300.

The first catch point that is a position the backward movement of the sliding block 1300 starts in the housing 1100 may be formed at a position where the sliding block 1300 sufficiently receives the tensile force from the first spring 1410.

Furthermore, the first catch point may be formed to have a first fixing protrusion easily inserted therein. Accordingly, when the sliding block 1300 returns to the first loading position, the sliding block 1300 may be automatically fixed by the first catch protrusion 1444 and the first catch point.

The first catch device 1440 may further include a first restoring spring 1446 that restores the positions of the first catch protrusion 1444 and the shoot button 1442 to a fixed state to automatically fix the sliding block 1300.

The sliding block 1300 that is released from the first catch device 1440 may move backward toward the flange block 1420, may release the second catch device 1450 that fixes the flange block 1420, and may guide the front movement of the flange block 1420.

The second catch device 1450 may be disposed at any one side surface of the housing 1100, may fix the flange block 1420 at a second loading position, and by removing the fixing, may shoot the loading unit 1400 and the outer needle 1220 forward by a compression force of the second spring 1430 toward the front portion of the housing 1100.

Although the second catch device 1450 is illustrated to be spaced apart from the surface where the first catch device 1440 is formed, as illustrated in FIG. 17, the second catch device 1450 may be formed on all surfaces where the loading unit 1400 is freely loaded and slide.

Furthermore, the second catch device 1450 may further include a second catch protrusion 1452 that protrudes to the inside of the housing 1100 and fixes the flange block.

The second catch protrusion 1452 may fix the flange block 1420 at the second loading position, and the flange block 1420 may have a second catch point at any one side surface corresponding to the second catch protrusion 1452 so that the second catch protrusion 1452 may firmly fix the flange block 1420.

The second catch point is a position where the flange block 1420 starts a forward movement in the housing 1100, and may be formed at a position where the flange block 1420 may be released as a contact surface of the sliding block 1300 arrives.

The second catch point may be formed such that the second catch protrusion 1452 is easily inserted. Accordingly, when the flange block 1420 returns to the second loading position, the flange block 1420 may be automatically fixed by the second catch protrusion 1452 and the second catch point.

The second catch device 1450 may further include a second restoring spring 1454 that returns the position of the second catch protrusion 1452 to automatically fix the flange block 1420, when the flange block 1420 returns to the second loading position.

In other words, the loading unit 1400 and the sliding block 1300 may be loaded by being fixed to the first catch device 1440 and the second catch device 1450 based on the loading operation of the loading handle 1500.

The tissue extraction method through the loading operation using the loading handle and the shooting operation according to one embodiment of the present disclosure is described below in detail with reference to the drawings.

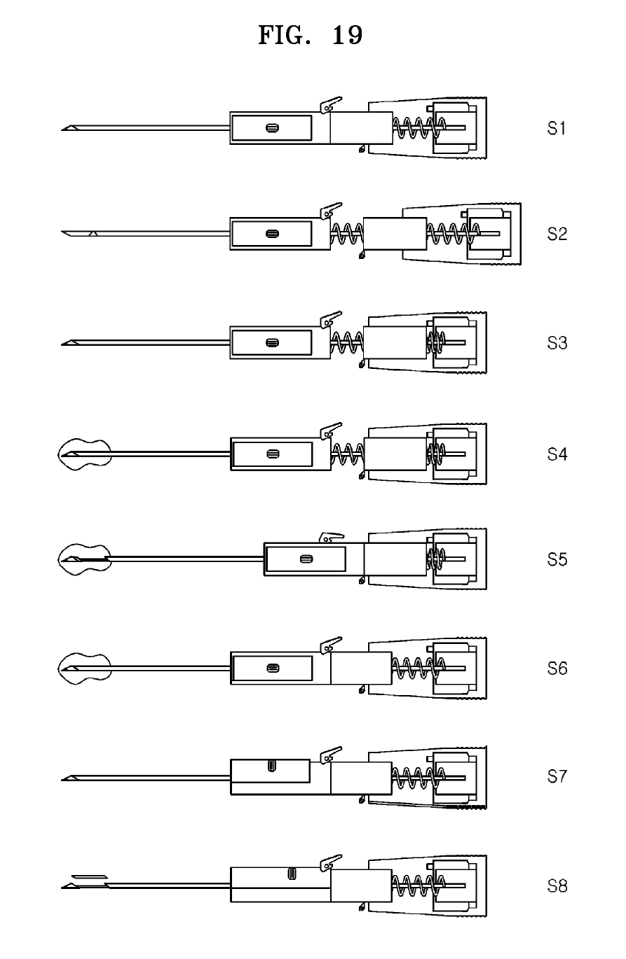

FIG. 19 illustrates a sequence of loading and operation of a cutting biopsy device according to an embodiment.

Referring to FIG. 19, when the outer needle 1220 advances forward covering the tissue extraction recess 1212 of the inner needle 1210, and the sliding block 1300 also arrives at the first loading position, the sliding block 1300 is fixed to the first catch device 1440 (S1).

The user pulls the loading handle 1500 to the rear side of the housing 1100, to fix the flange block 1420 to the second catch protrusion 1452, thereby performing the first loading (S2).

Next, when the first loading is completed, the user pushes the loading handle 1500 to the front side of the housing 1100 to compress and fix the second spring 1430, thereby performing the second loading (S3), and the loading operation is completed.

Next, when the loading operation is completed, the user inserts into a tissue to be extracted the needle set 1200 including the inner needle 1210 and the outer needle 1220 in a loaded state as above covering the tissue extraction recess 1212 of the inner needle 1210 (S4).

Next, when the loaded needle set 1200 arrives at the tissue to be extracted and a region of the tissue extraction recess 1212 sufficiently enters the tissue, the user may perform shooting of the outer needle 1220 by pressing the shoot button 1442 with a finger.

The shooting of the outer needle 1220 is described below in detail with reference to the drawings.

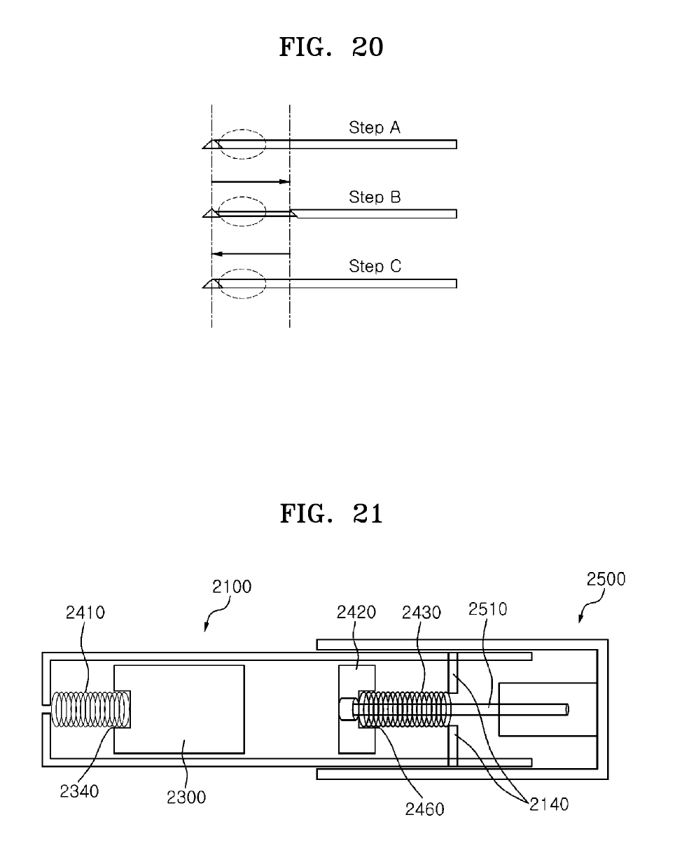

FIG. 20 illustrates a state of a leading end portion of a needle set according to an embodiment.

The shoot button 1442 pressed by an operator releases the first catch protrusion hinge-coupled with the shoot button 1442 from the first catch point (step A).

Accordingly, the fixing of the sliding block 1300 is removed and the sliding block 1300 is shot backward by the first spring 1410 and simultaneously the outer needle 1220 is also shot backward, and the tissue extraction recess 1212 covered by the outer needle 1220 is opened and thus a part of a specimen tissue is absorbed into the tissue extraction recess 1212 by a tissue pressure (step B). Furthermore, the sliding block 1300 that is shot backward has the second catch protrusion 1452 fixing the flange block 1420 released from the second catch point (S5).

The flange block 1420 fixed by the second catch protrusion 1452 is released and shot forward by the second spring 1430, and thus the sliding block 1300 is moved forward and accordingly, the outer needle 1220 is shot forward to cover the tissue extraction recess 1212 again (step C), and the cutting blade of the outer needle 1220 cuts the tissue accommodated in the tissue extraction recess 1212 (S6).

In this state, the tissue extraction recess 1212 receives a negative pressure from the first negative pressure space and the second negative pressure space formed in the front portion and the rear portion of the housing 1100, by the sliding block 1300, thereby having an effect of a high tissue extraction yield.

The sliding block 1300 and the flange block 1420 may be processed to remove an error in the lengths of the first spring 1410 located between the sliding block 1300 and the flange block 1420 and the second spring 1430 in the shooting operation. A first spring accommodation portion 1340 and a second spring accommodation portion 1460 may be formed in contact surfaces of the sliding block 1300 and the flange block 1420, respectively.

For example, as illustrated in FIGS. 3 and 15, in the sliding block 1300, the first spring accommodation portion 1340 for accommodating the first spring 1410 may be formed more inward than the contact surface of the sliding block 1300 contacting the flange block 1420.

In the flange block 1420, the second spring accommodation portion 1460 for accommodating the first spring 1410 may be formed more inward than the contact surface of the flange block 1420 contacting the sliding block 1300.

Furthermore, the flange block 1420 may include a third spring accommodation portion 1470 for accommodating the second spring 1430 in the rear portion thereof.

Accordingly, the loading units contact the sliding block 1300, the housing 1100, and the loading handle 1500 and reduce an operation distance error and enable a precise surgical procedure in performing the loading and shooting operations.

Furthermore, the disposable cutting biopsy device according to an embodiment may further include a shock absorbing member (not shown) on contact surfaces between the housing 1100, the sliding block 1300, the flange block 1420, and the loading handle 1500, and may reduce shock and noise occurring during a shooting operation by the first spring 1410 and the second spring 1430. The shock absorbing member may include rubber, urethane, or a shock absorbing material, but the present disclosure is not limited thereto.

A disposable cutting biopsy device according to another embodiment may include a housing having a sliding space formed therein, a needle set disposed in the housing and extracting a tissue sample by using an outer needle and an inner needle, a sliding block connected to the outer needle and capable of reciprocating back and forth in the sliding space of the housing, a loading unit that shoots the sliding block, and a loading handle disposed to be connected to the loading unit.

Furthermore, the disposable cutting biopsy device may further include a shock absorbing member on contact surfaces of the housing, the sliding block, the loading unit, and the loading handle.

As the housing, the needle set, and the shock absorbing member of the disposable cutting biopsy device according to another embodiment are substantially the same as those of the disposable cutting biopsy device according to the above one embodiment, detailed descriptions thereof are omitted (all except the spring structure and the loading operation are the same as those of the above one embodiment).

FIG. 21 is a cross-sectional view illustrating a combination of a housing, a loading unit, and a sliding block according to another embodiment.

As illustrated in FIG. 21, a loading unit is connected to a sliding block 2300 and disposed in a sliding space in a housing 2100.

For example, the loading unit 2400 may include a first spring 2410 connected to a front portion of the housing 2100 and a front portion of the sliding block 2300, a flange block 2420 disposed in the rear portion of the sliding block 2300, and a second spring 2430 having one side connected to the flange block 2420 and the other side connected to a rear portion of the housing 2100.

In this state, the first spring 2410 and the second spring 2430 are compression springs, the first spring 2410 may push the front portion of the housing 2100 and the sliding block 2300 away from each other, and the second spring 2430 may push the flange block 2420 and a loading handle 2500 away from each other.

The sliding block 2300 and the flange block 2420 are disposed in the housing 2100 and smoothly slide along the outer circumference of an inner needle, and may be disposed close to the housing 2100 without a gap therebetween.

For example, the sliding block 2300 and the flange block 2420 may be provided close to the housing 1100 without a gap therebetween to prevent air exchange therebetween so that, as the sliding block 2300 slides back and forth, a negative pressure or a positive pressure may be generated in a first negative pressure space formed in a front portion of the sliding block 2300 and a second negative pressure space formed in a rear portion of the sliding block 2300.

The loading unit 2400 may be connected to the loading handle 2500, as illustrated in FIG. 21, and may perform a loading operation.

The loading handle 2500 may be connected to the flange block 2420 to load the loading unit 2400.

The loading handle 2500 may surround the rear portion of the housing 2100, as illustrated in FIGS. 15 and 16. In particular, the loading handle 2500 may include a loading rod 2510 connected to the flange block 2420 so that the flange block 2420 may move backward according to the backward movement of the loading handle 2500. The loading rod 2510 may move backward as the flange block 2420 is caught by the loading rod 2510 when the loading handle 2500 moves backward. The loading rod 2510, as illustrated in FIG. 21, may penetrate the flange block 2420 in a lengthwise direction of the housing 2100, and may have a shape of a hook or a connecting rod, not a rod, but the present disclosure is not limited thereto.

The loading handle 2500 may be fixed after moving along a coupling guide formed in the housing 2100 to facilitate the loading operation. The loading handle 2500 may include a coupling protrusion that is fixed and moves along the coupling guide formed in the housing 2100.

As the coupling guide and the coupling protrusion are substantially the same as the coupling guide of the disposable cutting biopsy device according to an embodiment, a detailed description thereof is omitted.

In this state, a flange block support portion 2140 may be formed in the housing 2100 to be sufficiently long toward the inside of the housing 2100 to assist the loading of the second spring 2430.

In detail, as illustrated in FIG. 21, when the user pulls the loading handle 2500 backward, the flange block 2420 moves backward and the second spring 2430 is compressed. In this state, the flange block support portion 2140 may prevent the flange block 2420 from being detached out of the housing 2100, and may assist the compression of the second spring so that the second spring 2430 may be easily compressed.

The loading unit 2400 and the sliding block 2300 are loaded by a first catch device and a second catch device formed in the loading handle 2500 and the housing 2100 to be shot in the housing 2100 through restoration forces of the first spring 2410 and the second spring 2430, and accordingly the outer needle is also loaded.

The sliding block 2300 and the flange block 2420 are loaded by being fixed by the first catch device and second catch device not to be operated by a force transferred from the first spring 2410 and the second spring 2430, and the loading unit 2400, the sliding block 2300, and the outer needle are shot by releasing the first catch device and the second catch device.

The first catch device may be disposed at any one side surface of the housing 2100, may fix the sliding block 2300 at a first loading position, and by removing the fixing, may shoot the loading unit 2400 and the outer needle backward toward the flange block 2420 by a compression force of the first spring 2410.

The first catch device may include a shoot button, and the user may start a shooting operation of the outer needle by removing the fixing of the sliding block 2300 simply by pressing the shoot button.

Furthermore, the first catch device may further include a first catch protrusion that is hinge-coupled to the shoot button and protrudes inward to fix the sliding block 2300.

The first catch protrusion may have the sliding block 2300 fixed at a loading position, and the sliding block 2300

may have a first catch point at any one side surface corresponding to the first catch protrusion so that the first catch protrusion may firmly fix the sliding block 2300.

The first catch point that is a position where the backward movement of the sliding block 2300 starts in the housing 2100 may be formed at a position where the sliding block 2300 sufficiently receives the compression force from the first spring 2410.

Furthermore, the first catch point may be formed to have a first fixing protrusion easily inserted therein. Accordingly, when the sliding block 2300 returns to the first loading position, the sliding block 2300 may be automatically fixed by the first catch protrusion and the first catch point.

The first catch device may further include a first restoring spring that restores the positions of the first catch protrusion and the shoot button to a fixed state to automatically fix the sliding block 2300 when the sliding block 2300 returns to the first loading position.

The sliding block 2300 that is released from the first catch device may move backward toward the flange block 2420, may release the second catch device that fixes the flange block 2420, and may guide the forward movement of the flange block 2420.

The second catch device may be disposed at any one side surface of the housing 2100, may fix the flange block 2420 at a second loading position, and by removing the fixing, may shoot the loading unit 2400 and the outer needle forward by a compression force of the second spring 2430 toward the front portion of the housing 2100.

Although the second catch device is illustrated to be spaced apart from the surface where the first catch device is formed, the second catch device may be formed on all surfaces where the loading unit 2400 is freely loaded and is slidable.

Furthermore, the second catch device may further include a second catch protrusion that protrudes to the inside of the housing and fixes the flange block 2420.

The second catch protrusion may fix the flange block 2420 at the second loading position, and the flange block 2420 may have a second catch point at any one side surface corresponding to the coupling protrusion so that the second catch protrusion may firmly fix the flange block 2420.

The second catch point is a position where the forward movement of the flange block 2420 starts in the housing 2100, and may be formed at a position where the flange block 2420 may be released as a contact surface of the sliding block 2300 arrives.

The second catch point may be formed such that the second fixing protrusion is easily inserted. Accordingly, when the flange block 2420 returns to the second loading position, the flange block 2420 may be automatically fixed by the second catch protrusion and the second catch point.

The second catch device may further include a second restoring spring that returns the position of the second catch protrusion to automatically fix the flange block 2420, when the flange block 2420 returns to the second loading position.

In other words, the loading unit 2400 and the sliding block 2300 may be loaded by being fixed to the first catch device and the second catch device based on the loading operation of the loading handle 2500.

The tissue extraction method through the loading operation using the loading handle 2500 and the loading unit 2400 and the shooting operation according thereto is described below in detail with reference to the drawings.

At the initial state, in the sliding block 2300 and the first spring 2410, as an elastic force of the second spring 2430 is greater than an elastic force of the first spring 2410, the flange block 2420 compresses the first spring 2410 and the sliding block 2300 by the second spring 2430, which is a first loading state that is a loaded state.

As the user pulls backward the flange block 2420 that loaded the sliding block 2300 by using the loading handle 2500, the second spring 2430 is backward compressed by using the flange block support portion 2140, and the flange block 2420 is fixed to the second catch device, thereby completing the second loading.

Next, when the loading operation is completed, the user inserts the needle set including the inner needle and the outer needle covering the tissue extraction recess of the inner needle in a loaded state into a tissue to be extracted.

Next, when the loaded needle set arrives at the tissue that the user desires to extract and a region of the tissue extraction recess is sufficiently inserted, the user presses the first shoot button with a finger to perform a shooting operation of the outer needle.

The first shoot button pressed by the operator may release the first catch protrusion hinge-coupled to the first shoot button from the first catch point. Accordingly, the fixing of the sliding block 2300 is removed and shot backward by the first spring 2410 and simultaneously the outer needle is shot backward as well, and the tissue extraction recess covered by the outer needle is opened so that a part of a specimen tissue is rapidly absorbed in the tissue extraction recess by a tissue pressure. Furthermore, the sliding block 2300 that is shot backward causes the second catch protrusion fixing the flange block 2420 to be released from the second catch point.

The flange block 2420 fixed by the second coupling protrusion is released and shot forward by the second spring 2430 and pushes the sliding block 2300. Accordingly, the outer needle is shot forward to cover the tissue extraction recess again, and a cutting blade of the outer needle cuts the tissue accommodated in the tissue extraction recess.

The sliding block 2300 and the flange block 2420 may be processed to remove an error in the lengths of the first spring 2410 and the second spring 2430 located between the sliding block 2300 and the flange block 2420 in the shooting operation. A first spring accommodation portion 2340 may be formed in a region where the sliding block 2300 and the first spring 2410 are in contact with each other. For example, as illustrated in FIG. 21, in the sliding block 2300, the first spring accommodation portion 2340 for accommodating the first spring 2410 may be formed more inward than the contact surface of the sliding block 2300 contacting the first spring 2410. In the flange block 2420, a second spring accommodation portion 2460 for accommodating the second spring 2430 may be formed more inward than the contact surface of the flange block 2420 contacting the second spring 2430.

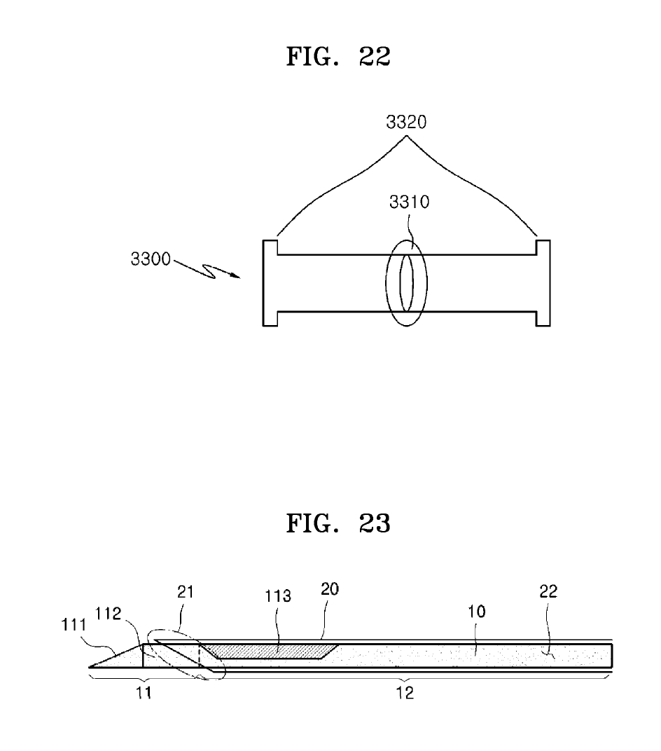

FIG. 22 is a side view of a sliding block according to another embodiment sliding block.

As illustrated in FIG. 22, in the sliding space of the housing, to reduce friction with the sliding space and keep sealing during sliding of a sliding block 3300, the sliding block 3300 may include, as illustrated in FIG. 22, at least one O-ring 3310 in a body portion of the sliding block 3300.

Furthermore, to prevent the O-ring 3310 from being detached as the sliding block 3300 slides back and forth, detachment prevention steps 3320 may be further provided at the front and rear portions of the sliding block.

FIG. 23 is a side cross-sectional view of a structure of a needle of the cutting biopsy device according to the related art.

Referring to FIG. 23, in a cutting biopsy method, a needle may include a needle set of an inner needle 10 and an outer needle 20. The inner needle 10 may include a head part 11 and a body part 12, and the outer needle 20 may include a cutting blade 21 and a needle tube 22 therein. A tissue extraction recess 113 is formed in the body part 12 of the inner needle 10, and the head part 11 may include a leading tip 111 and an extension portion 112.

As illustrated in FIG. 23, the inner needle 10 extends from the leading tip 111 in a lengthwise direction to the extension portion 112 and the body part 12, and the tissue extraction recess 113 is formed in an area on an upper surface of the body part 12, in detail, in an area in a direction close to the leading tip 111. For the outer needle 20, the cutting blade 21 that is inclined is formed in one end surface in a direction close to the leading tip 111, and is configured to be capable of a relative motion in a lengthwise direction with respect to the inner needle 10 through the needle tube 22.

In the cutting biopsy device according to the related art having such as needle shape, when a structure of the inner needle 10 and the outer needle 20 assembled with each other enters the human body of a tissue extraction region, and arrives at a tissue extraction region, the inner needle 10 is first shot in the lengthwise direction by performing a linear motion, and then the outer needle 20 is shot in a shot direction of the inner needle 10, and through a dual advancement method of the inner needle 10 and the outer needle 20, the cutting blade 21 cuts a tissue and performs cutting extraction of a specimen.

In this case, as the leading tip 111 of the inner needle 10 has a blade shape, when there is a major tissue such as artery, gallbladder, or nerves in the rear of a test tissue or it is applied to a pediatric child having a small body, it may be a problem that only a minor deviation in an aim direction and a rear safe distance prediction may cause a damage to the major tissue because a shot inner needle cannot be stopped.

As the cutting blade 21 of the outer needle 20 protrudes further outward than the outer surface of the inner needle 10, the thickness of the needle relatively increases and thus a surgical procedure becomes difficult, and the tissue is moved backward by the cutting blade 21 or damaged when the needle set enters the tissue, thereby causing secondary damage.

Furthermore, when the inner needle 10 is shot, as the diameter of the inner needle 10 is small and the leading tip 111 is inclined only in one direction, the inner needle 10 advancing forward may not penetrate through a dense membrane tissue or a hard calcified tissue or may be bent, and thus a nodule may be pushed backward or the inner needle may not reach an accurate extraction target tissue region, so that a surgical procedure becomes very difficult.

Furthermore, due to the existence of the extension portion 112 of the inner needle 10, the accurate extraction of a desired tissue is difficult, and thus an accurate surgical procedure may be difficult.

In order to enable the needle set to accurately arrive at the above-described extraction target tissue, technology about improvement of accuracy, such as the use of ultrasonic guide equipment, has been developed much. However, in the above-described cutting biopsy device, as a risk tissue may exist in the rear of a specimen to be extracted, it is very important to identify the location of a tip point, that is, a tip point of the leading tip 111. However, as described above, due to the existence of the extension portion 112 and the characteristics of a blade shape of the leading tip 111, a surgical procedure has been carried out with risk or has not been carried out due to risk.

Accordingly, one embodiment of the present disclosure is invented to solve the above-described problem of the related art by providing a new shape of the leading tip 111 of the inner needle 10 to prevent damage to a major tissue.

It is another purpose of the present disclosure to further decrease the thickness of the outer needle 20 or newly propose a thickness relation with respect to the inner needle 10, thereby decreasing the thickness of the needle, so that the size of an injury of an affected part may be reduced and the damage to a tissue by the cutting blade 21 may be reduced.

Furthermore, it is another purpose of the present disclosure to greatly improve accuracy of tissue extraction by reducing the length of the extension portion 112 of the inner needle 10.

Furthermore, it is another purpose of the present disclosure to greatly improve accuracy of a surgical procedure by providing a structure such as the leading tip 111 and the outer needle 20 to identify an accurate location of the leading tip 111 when using an ultrasonic guide.

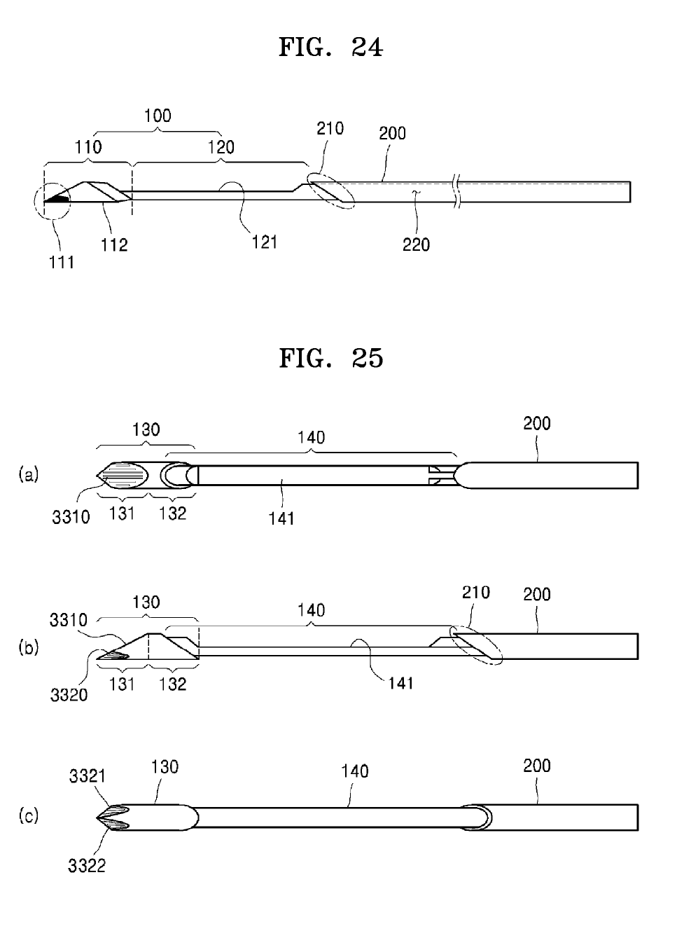

FIG. 24 is a side view of a structure of a needle of a cutting biopsy device according to a first embodiment of the present disclosure.

Referring to FIG. 24, in a structure of a needle of the cutting biopsy device according to an embodiment of the present disclosure, a needle set may include an inner needle 100 and an outer needle 200.

The inner needle may mean a configuration having an assigned length in the back and forth direction, and including a head part 110 having the leading tip 111 formed thereon to penetrate and enter at least a biological tissue and a body part 120 extending in a lengthwise direction from the head part 110 through the extension portion 112 of the head part 110 and having a tissue extraction recess 121 for accommodating a biological tissue formed to be concave therein.

The back and forth direction may mean a direction in which the needle set enters the body, and may signify the same direction as a lengthwise direction described below. The leading tip 111 may mean an area where a sharp needle is formed in the cutting biopsy device.

The extension portion 112, which is an area of the head part 110 connected to the body part 120, may mean, for example, an area of the head part 110 except an area where the leading tip 111 is formed. As illustrated in FIG. 24, the extension portion 112 may form an inclined outer surface having a thickness changed for connection with the body part 120.

The tissue extraction recess 121 may mean an area of the outer needle 200, where a biological tissue is cut by a cutting blade 210 of the outer needle 200 and accommodated based on a relative motion to the inner needle 100. The tissue extraction recess 121 may adopt any shape having a concave cavity for accommodation of a biological tissue.

The body part 120 may mean a configuration including, for example, the tissue extraction recess 121, as described above, and having a bar shape of a certain needle, to be included in a needle tube 220 of the outer needle 200, and capable of a relative motion to the outer needle 200.

In this state, to reduce the thickness of the outer needle 200, in an area of the head part 110, a thickness of an area except an area where the leading tip 111, that is, first to third inclined surfaces described below, is formed, that is, the maximum outer diameter of the body part 120, may be less than the maximum outer diameter of the head part 110.

In this state, the thickness of the outer needle 200, that is, a distance from the center of the outer needle 200 to an outermost surface of the outer needle 200, that is, the radius of the outer needle 200, may be identical to a distance from the center of the head part 110 to an area except an area where the first inclined surface, the second inclined surface, and the third inclined surface are formed, that is, to an outermost surface of an area corresponding to the extension portion 112. In other words, the outer diameter of the outer needle 200 may be formed identical to the maximum outer diameter of the head part 110 of the inner needle 100.

In other words, the maximum outer diameter of the head part 110 of the inner needle 100 may match the outer diameter of the outer needle 200 (the maximum outer diameter), and thus, as a whole, the inner needle 100 and the outer needle 200 may constitute one needle. In other words, in the outer surface, it may be a core feature of other first embodiment of the present disclosure to configure the inner needle 100 and the outer needle 200 without a step therebetween.

In other words, as described above, when a biological tissue is cut and accommodated in a method in which the inner needle first advances and then the outer needle advances, if the inner needle is formed to be thin, a penetration force of the inner needle may be reduced or the inner needle is bent while advancing due to the thin thickness of the inner needle. Accordingly, there is a limit to the reduction of the thickness of the inner needle, and the outer needle may also have a thick structure to protrude from the outer surface of the inner needle. According to the first embodiment and a second embodiment to be described below of the present disclosure, it is the characteristics of the above-described relative motion that, after the inner needle 100 and the outer needle 200 together enter a tissue, the inner needle 100 is fixed and only the outer needle 200 retreats and advances. Accordingly, there is no need of the inner needle 100 only advancing, and the limitation in the thickness of the inner needle 100 may disappear.

Accordingly, the thickness of the inner needle 100 may be designed to be thinner, and accordingly the thickness of the outer needle 200 may be configured to be identical to the inner needle 100, as described above. Furthermore, as described above, as the bending of the inner needle 100 or the difficulty in the penetration of a tissue is addressed, a phenomenon that a nodule is pushed backward or the inner needle 100 fails to arrive at an accurate extraction target tissue region may be reduced or prevented.

Furthermore, the diameter of the inner needle 100 may be further decreased and the thickness of the outer needle 200 may be further decreased, based on a reciprocating motion structure of the above-described the outer needle 200. In detail, as described above, the maximum outer diameter of the head part 110 of the inner needle 100 matches the outer diameter of the outer needle 200 (for example, the maximum outer diameter), and as a whole, the inner needle 100 and the outer needle 200 may be configured to be one needle. In other words, in the outer surface, as the inner needle 100 and the outer needle 200 are configured without a step therebetween, the thickness of a needle may be reduced and thus a surgical procedure may be performed easily and accurately, and smooth insertion may be possible so that patient's pain and the extension of an injury of an affected part may be reduced.

In the first embodiment of the present disclosure, the above-described characteristics of the relative motion between the outer needle 200 and the inner needle 100, that is, the backward and forward motion characteristics of the outer needle 200, may be used for a dual advancement method of the above-described according to the related art based on a degree of processing when the thickness of the needle set is equal to or less than 16G.

Accordingly, the above-described characteristics of the relative motion between the outer needle 200 and the inner needle 100 may be understood to be differently applied based on the thickness of a needle. The leading tip 111 may means a region having a sharp needle shape in the configuration of the inner needle 100. The characteristic configuration of the leading tip in the present disclosure is described with reference to FIGS. 24, 26, 27, and 28.

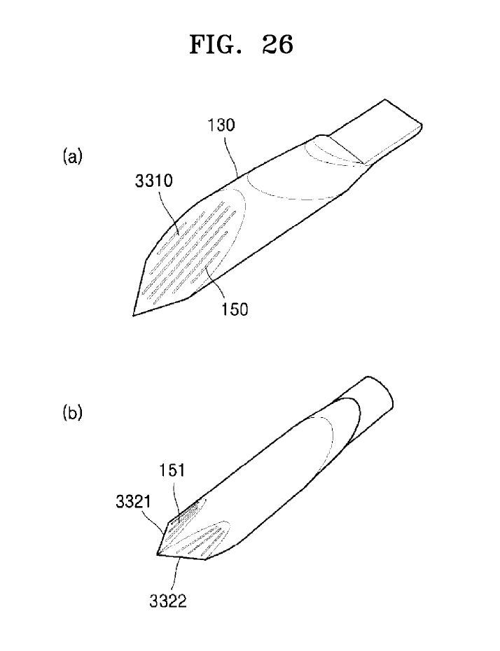

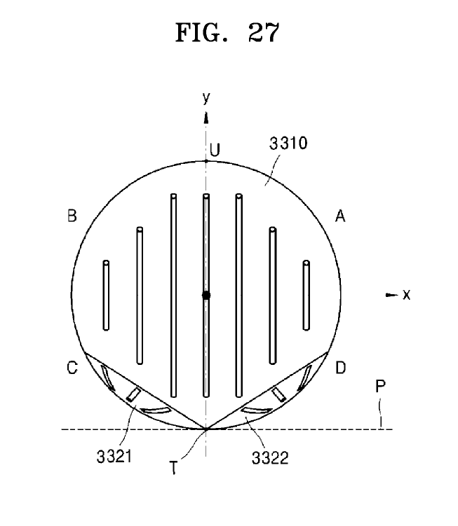

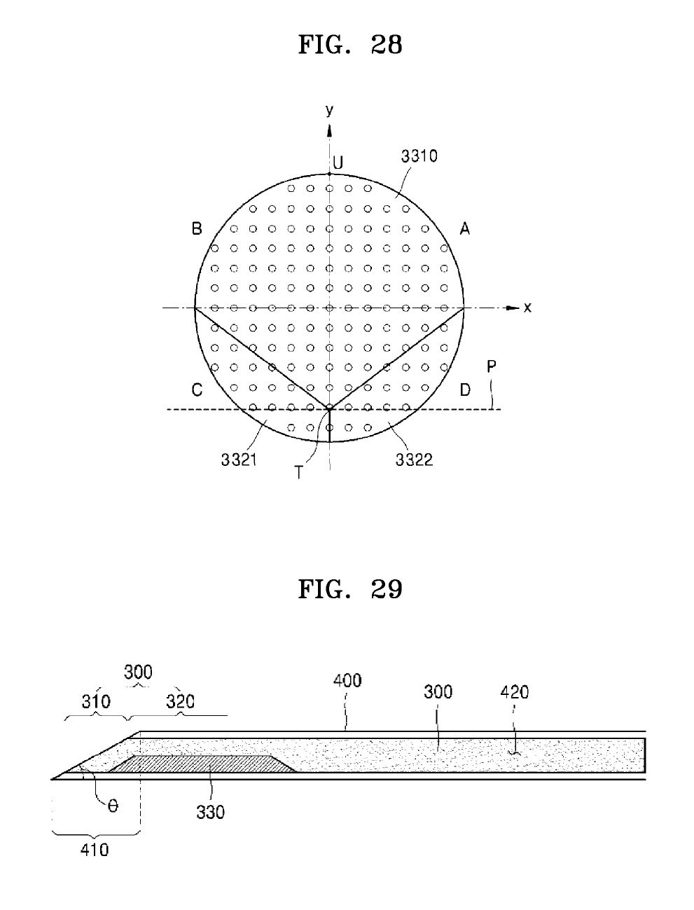

FIGS. 26A and 26B are perspective views for describing a structure of an inner needle of a cutting biopsy device according to a first embodiment of the present disclosure. FIGS. 27 and 28 are front views for describing a structure of a leading tip of a cutting biopsy device according to a first embodiment of the present disclosure.

Referring to FIGS. 27 and 28 first, in the first embodiment of the present disclosure, the front cross-section of the leading tip may be divided into four quadrants A, B, C, and D, in which the upper right quadrant is set to a first quadrant A, and the other quadrants counterclockwise from the first quadrant A may be set to a second quadrant B, a third quadrant C, and a fourth quadrant D.

In this state, in an area corresponding to the first and second quadrants A and B, as in the structure of the needle according to the related art, a first inclined surface 3310 is formed to face one surface P in the lengthwise direction including a tip point T that is one point in an axis direction of the inner needle (no reference numeral, for example, a center axis of a needle).

In an optional embodiment, the first inclined surface formed in each of the first and second quadrants A and B may be connected to each other having one plane shape.

It may be seen that a second inclined surface 3321 is formed in a partial area of an area corresponding to the third quadrant C to face the tip point T, and that a third inclined surface 3322 is formed in a partial area of an area corresponding to the fourth quadrant D to face the tip point T.

It may be seen from the above structure and referring to FIGS. 27 and 28 that the first inclined surface 3310, the second inclined surface 3321, and the third inclined surface 3322 are formed such that a point where the first inclined surface 3310, the second inclined surface 3321, and the third inclined surface 3322 simultaneously meet together is the tip point T.

In other words, from the first inclined surface 3310, the second inclined surface 3321 and the third inclined surface 3322, it may be seen that the configuration of the leading tip is not that of a cutting surface of a general needle, but that of a sharp structure having the tip point T as a center like an awl.

In this state, as can be seen from comparison between FIG. 27 and FIG. 28, the tip point T and the one surface P including the tip point T may be formed in contact with an outer surface of a circle as illustrated in FIG. 27 with respect to an y axis, or located inside a circle depending on a degree of processing.

In other words, according to a degree of back cutting of the second inclined surface 3321 and the third inclined surface 3322, the tip point T may be located within acceptable limits vertically in the y-axis direction. Accordingly, the one surface P including the tip point T may move vertically and the acceptable limit is 1/2 of the radius.

According to the above configuration, in a general inclination structure of the leading tip of an inner needle, the structure of the leading tip having a sharp shape like an awl with the tip point T as a center is formed by back-cutting the third and fourth quadrants C and D.

Accordingly, when the needle enters a tissue, the damage to the major tissue due to the general inclination needle structure may be reduced. Additionally, according to the above-described structure, when the needle enters a tissue, the second and third inclined surfaces 3321 and 3322 may offset the application of a load downward to the needle by hard tissues with a load applied upward, and thus the bending of a needle during entering may be reduced.

Additionally, referring to FIGS. 27 and 28, it may be seen that the first inclined surface 3310 is a surface that makes the one surface P in the lengthwise direction including the tip point T that is one point in the axis direction of the inner needle parallel to the axis of the inner needle and orthogonal to the direction toward a top point U of the inner needle, and is inclined to the one surface P to meet the one surface P at the leading tip.

As illustrated in FIGS. 27 and 26, the second and third inclined surfaces 3321 and 3322 may be symmetric to the left and right with respect to the tip point T as a center.

Based on the above, referring to FIGS. 26A and 26B, as described above, the first inclined surface 3310, the second inclined surface 3321, and the third inclined surface 3322 are formed in a leading tip region of a head part 130.

In this state, in an optional embodiment, engraved patterns 150 and 151 may be formed on the first inclined surface 3310, the second inclined surface 3321, and the third inclined surface 3322. The engraved patterns 150 and 151 may include various patterns, for example, random scratches, dimples, and line-type engraved patterns.

In the present disclosure, the engraved patterns 150 and 151 amplify an echo phenomenon of ultrasound to generate a strong signal when the location of a leading tip is identified by using an ultrasonic guide, thereby strongly indicating the location of the leading tip in the ultrasonic guide.

In the related art, the configuration of a leading tip has a blade shape like a cutting blade of a needle. Accordingly, even when an engraved pattern is formed as in the present disclosure and echo is amplified thereby, amplification is generated in a line shape, not in a point shape, and thus the location of a point where the leading tip is located could not accurately identified. Accordingly, as a risk tissue may exist in the rear of a specimen to be extracted, although it is important to identify the location of a tip point, a surgical procedure has been carried out with risk or the surgical procedure has not been carried out.

However, according to the above-described embodiment, the needle has a structure such that the first inclined surface 3310, the second inclined surface 3321, and the third inclined surface 3322 are formed and meet at the tip point. In an optional embodiment, as the engraved patterns 150 and 151 are formed in the first inclined surface 3310, the second inclined surface 3321, and the third inclined surface 3322 as above described, echo is amplified with the tip point as a center. Accordingly, as amplification occurs around a point, the location of a tip point may be accurately identified. Accordingly, compared with the related art in which the location of a leading tip is identified by using the ultrasonic guide, the identification of the location of the tip point becomes more precise, and thus the risk in a surgical procedure due to a possibility of existence of a risk tissue in the rear of a specimen may be reduced.

FIGS. 25A, 25B, and 25C are respectively a plan view, a side view, and a bottom view for describing a modified example of a structure of a needle of a cutting biopsy device according to a first embodiment of the present disclosure. In the following description, redundant descriptions with those of FIGS. 24 and 26 to 28 are omitted.

Referring to FIG. 25, in a modified example of the first embodiment of the present disclosure, it may be seen that a configuration of an extension portion 132 has a slight difference while the other configurations are identical.

In other words, in the extension portion 112 of FIG. 24, the leading tip 111 is connected to the body part 120 and the thickness thereof gradually decreases, whereas the extension portion 132 of FIGS. 25A, 25B, and 25C has a constant thickness in a region connected to a leading tip 131 and then is inclined in a region adjacent to a body part 140 and the thickness thereof decreases.

The above-described configuration of FIG. 24 and FIGS. 25A, 25B, and 25C may be optionally implemented.

Referring to FIGS. 25A, 25B, and 25C, it may be seen that the first inclined surface 3310 is formed at the leading tip 131, and the head part 130 may include the extension portion 132. A tissue extraction recess 141 is formed in the body part 140, as described above, and then the body part 140 is accommodated in a needle tube of the outer needle 200. The outer needle 200, as illustrated, may include the cutting blade 210, and a biological tissue is cut based on the forward and backward motion of the outer needle 200 and accommodated in the tissue extraction recess 141 with a seal.

A configuration of an auxiliary inclined surface 3320 including the second and third inclined surfaces 3321 and 3322 is formed by back-cutting with respect to the first inclined surface 3310, as illustrated in FIGS. 25B and 25C.

In this state, as illustrated in FIG. 25, the length of the first inclined surface 3310 in the lengthwise direction may be greater than the length of the second and third inclined surfaces 3321 and 3322 in the lengthwise direction.

FIG. 29 is a side cross-sectional view of a structure of a needle of a cutting biopsy device according a second embodiment of the present disclosure.

Referring to FIG. 29, a structure of a needle of the cutting biopsy device according another embodiment of the present disclosure may also include an inner needle 300 and an outer needle 400.

In this state, unlike the first embodiment, the inner needle 300 may include a leading tip 310 that enters a biological tissue by penetrating the same and a body part 320. In this state, a tissue extraction recess 330 for accommodating a biological tissue is characteristically formed on a lower surface of a region adjacent to an area including the leading tip 310 and the body part 320, in detail, an area where the leading tip 310 is formed.

In general, the tissue extraction recess 330 is formed on an upper surface of the inner needle 300, which is forced by the characteristics (dual advancement) of the relative motion of the outer needle and the inner needle according to the above-described related art. In such a structure, the tissue extraction recess 330 should start at least from a region where the leading tip 310 ends. In other words, at least the extension portion is needed as illustrated in the drawing regarding the first embodiment of the present disclosure.

However, in another embodiment of the present disclosure, as described above, as the outer needle 400 has the characteristics of a relative motion, that is, the outer needle 400 enters a tissue with the inner needle 300 and retreats and advance by the manipulation of an operator, as described above, the tissue extraction recess 330 may be located on the lower surface of a region adjacent to the area where the leading tip 310 is formed.

This is because, when the tissue extraction recess 330 is located on the lower surface, the biological tissue may be accommodated, due to the characteristics thereof, on a surface of the tissue extraction recess 330, by a tissue pressure of a living body. According to the characteristics of the relative backward and forward motion of the above-described the outer needle 400, there is no difficulty extracting a tissue. In particular, in the description of FIG. 29, as described below, in another embodiment of the present disclosure, a negative pressure structure is formed to strongly absorb a biological tissue to the tissue extraction recess 330, and thus even when the tissue extraction recess 330 is located on the lower surface of the inner needle 300, the extraction of a tissue may be efficiently performed.

As illustrated in FIG. 29, according to another embodiment of the present disclosure, when the tissue extraction recess 330 is located on the lower surface of the inner needle 300, the configuration of an extension portion is not needed, and in particular, as illustrated in FIG. 29, the tissue extraction recess 330 may be formed in the region of the leading tip 310, and thus an interval between the leading tip 310 and the tissue extraction recess 330 may be reduced. Then, when using an ultrasonic guide, the tissue extraction recess 330 may be accurately positioned at the location of a biological tissue to be extracted, thereby greatly improving precision of the tissue extraction. As described above, risk of a surgical procedure due to a risk tissue that may be located in the rear of a specimen may be reduced and impossibility of a surgical procedure may be reduced.

In an optional embodiment, the tissue extraction recess 330 may overlap the leading end tip 310 in at least one region.

The outer needle 400 performs a similar function to the first embodiment of the present disclosure. In other words, the outer needle 400 includes the inner needle 300 in a needle tube 420, and performs a relative motion, in detail, moving in the lengthwise direction in a direction opposite to the leading tip 310 of the inner needle 300 and again in the direction toward the leading tip 310 of the inner needle 300 by the manipulation of an operator, and thus a biological tissue may be accommodated in the tissue extraction recess 330 with a seal.

In this state, as the tissue extraction recess 330 is formed on the lower surface of the inner needle 300, a region 410 where a cutting blade of the outer needle 400 is formed may have the same inclination as that of the leading tip 310 of the inner needle 300. In other words, the region 410 where the cutting blade of the outer needle 400 formed may have an inclined surface such that the inner needle 300 and the outer needle 400 have the same inclination degree (θ) and an inclined surface formed by the leading tip 310 of the inner needle 300 has an extended shape as illustrated in FIG. 29. In other words, the inner needle 300 and the outer needle 400 each have an inclined surface in the same direction.

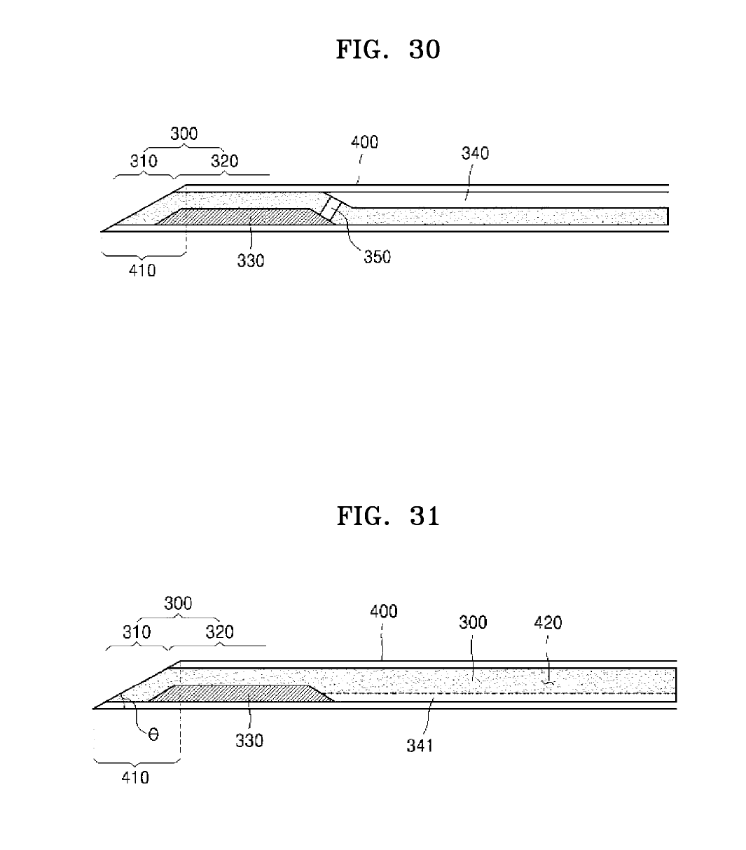

FIGS. 30 and 31 are side cross-sectional views of a structure of a needle of a cutting biopsy device according to a modified example of a second embodiment of the present disclosure.

Referring to FIG. 30 first, the basic structure of FIG. 30 is the same as the structure illustrated in FIG. 29.

In other words, the inner needle 300 may include the leading tip 310 and the body part 320 that enter a biological tissue by penetrating the same. In this state, the tissue extraction recess 330 for accommodating a biological tissue is formed on the lower surface of the region adjacent to the area including the leading tip 310 and the body part 320, in detail, the area where the leading tip 310 is formed. The outer needle 400 includes the inner needle 300 in the needle tube 420, and moves in the lengthwise direction in a direction opposite to the leading tip 310 of the inner needle 300 and again in the direction toward the leading tip 310 of the inner needle 300 by the manipulation of an operator, and thus a biological tissue may be accommodated in the tissue extraction recess 330 with a seal.

Furthermore, the region 410 where the cutting blade of the outer needle 400 is formed may have the same inclination degree as that of the leading tip 310 of the inner needle 300. In other words, the region 410 where the cutting blade of the outer needle 400 is formed may be formed such that the inclined surface on which the leading tip 310 of the inner needle 300 is formed may extend as illustrated in FIG. 29.

In the modified example of another embodiment illustrated in FIG. 30, a negative pressure portion 340 may be formed in the inner needle 300. The negative pressure portion 340 may be formed in a region at one side in a direction opposite to the leading tip 310 among an area adjacent to an area where the tissue extraction recess 330 is formed, may be formed concave so that air may flow through the tissue extraction recess 330 and a hole 350, and may perform a function of lowering air pressure of the tissue extraction recess 330.

In other words, when the outer needle 400 moves, the negative pressure portion 340 is formed to have a very low air pressure by using various methods such as a vacuum induction method or a separate vacuum induction apparatus. By using the same, the air pressure of the tissue extraction recess 330 is lowered, and thus a force exerting toward the tissue extraction recess 330 from the outside is generated. In this case, the biological tissue may be further strongly absorbed in the tissue extraction recess 330, thereby greatly increasing efficiency of extraction.

Referring to FIG. 31, the basic structure of FIG. 31 is the same as the structures illustrated in FIGS. 29 and 30. In other words, the inner needle 300 may include the leading tip 310 and the body part 320 that enter a biological tissue by penetrating the same. In this state, the tissue extraction recess 330 for accommodating a biological tissue is formed on the lower surface of the region adjacent to the area including the leading tip 310 and the body part 320, in detail, the area where the leading tip 310 is formed. The outer needle 400 includes the inner needle 300 in the needle tube 420, and moves in the lengthwise direction in a direction opposite to the leading tip 310 of the inner needle 300 and again in the direction toward the leading tip 310 of the inner needle 300 by the manipulation of an operator, and thus a biological tissue may be accommodated in the tissue extraction recess 330 with a seal.

Furthermore, the region 410 where the cutting blade of the outer needle 400 is formed may have the same inclination degree as that of the leading tip 310 of the inner needle 300. In other words, the region 410 where the cutting blade of the outer needle 400 is formed may be formed such that the inclined surface on which the leading tip 310 of the inner needle 300 is formed may extend as illustrated in FIG. 29.

In a modified example of another embodiment illustrated in FIG. 31, a negative pressure portion 341 of a different shape from that of FIG. 30 may be formed the inner needle 300. The negative pressure portion 341 is formed in a region at one side opposite to a direction toward the leading tip 310 among the region adjacent to the area where the tissue extraction recess 330 is formed. As illustrated in FIG. 31, the negative pressure portion 341 is connected to the tissue extraction recess 330 in the form of a groove, and may perform a function of lowering the air pressure of the tissue extraction recess 330.

In other words, when the outer needle 400 moves, the negative pressure portion 341 is formed to have a very low air pressure by using various methods such as a vacuum induction method or a separate vacuum induction apparatus. By using the same, the air pressure of the tissue extraction recess 330 is lowered, and thus a force exerting toward the tissue extraction recess 330 from the outside is generated. In this case, the biological tissue may be further strongly absorbed in the tissue extraction recess 300, thereby greatly increasing efficiency of extraction.

The one or more embodiments described above are intended to exemplify the main concepts of the present disclosure, and not limit the present disclosure. It will be understood by one of ordinary skill in the art that various substitutions, amendments, or modifications may be made to the one or more embodiments of the present disclosure without departing from the spirit and scope of the present disclosure.

### INDUSTRIAL APPLICABILITY

As such, according to the disposable cutting biopsy device of the present disclosure, while in the disposable biopsy device according to the related art application of a negative pressure is difficult or even when applied, a negative pressure may be used only one time through a movement in one direction within an assigned negative pressure space, in the present disclosure, as the sliding block connected to the outer needle performs a reciprocating motion, a negative pressure space is formed two times sequentially in the front and rear portions of the housing, and thus pressure is generated in two steps. Consequently, tissue extraction yield may be greatly improved through the negative pressure mechanism in which the negative pressure is sufficiently applied to the entire process from starting the accommodation of a tissue to be extracted and completing the accommodation to the completion of tissue cutting.

Furthermore, as the negative pressure mechanism alternately uses empty spaces in opposite directions formed in the reciprocating motion of the sliding block closely disposed in the housing, a negative pressure using the negative pressure space may be generated by a simple method and thus the purpose of the disposable cutting biopsy device is met.

Furthermore, as the sliding block includes the mother block and the baby block, and the mother block and the baby block are assembled and disassembled by manipulating a lever button formed in the baby block, the extraction of a cut tissue may be possible with a simple operation.

1. A disposable cutting biopsy device comprising:
a housing formed in a tube shape and having a sliding space therein;
a needle set disposed in the housing through a front portion hole of the housing and comprising an inner needle having a tissue extraction recess to extract a tissue and an outer needle having a blade formed at a leading end to cut the tissue accommodated in the tissue extraction recess and performing a reciprocating motion along an outer circumference of the inner needle in a form of surrounding the inner needle;
a sliding block connected to the outer needle and disposed to perform a reciprocating motion back and forth in the sliding space;
a loading unit connected to the sliding block and shooting the sliding block; and
a loading handle disposed to be connected to the loading unit,
wherein, when the sliding block moves backward, a first negative pressure space is formed in a front portion of the sliding space, and
when the sliding block moves forward, a second negative pressure space is formed in a rear portion of the sliding space.

2. The disposable cutting biopsy device of claim 1, wherein
a negative pressure from the first negative pressure space and the second negative pressure space is applied to the tissue extraction recess.

3. The disposable cutting biopsy device of claim 1, wherein the housing further comprises:
a first positive pressure valve formed in a front portion of the housing to remove a positive pressure formed in the first negative pressure space as the sliding block moves forward; and
a second positive pressure valve formed in a rear portion of the housing to remove a positive pressure formed in the second negative pressure space as the sliding block moves backward.

4. The disposable cutting biopsy device of claim 3, wherein,
when the sliding block moves backward, the first positive pressure valve is closed and a negative pressure is formed in the first negative pressure space, and the second positive pressure valve is opened and a positive pressure of the second negative pressure space is removed, and
when the sliding block moves forward, the first positive pressure valve is opened and a positive pressure of the first negative pressure space is removed, and the second positive pressure valve is closed and a negative pressure is formed in the second negative pressure space.

5. The disposable cutting biopsy device of claim 1, wherein
the inner needle is formed such that the tissue extraction recess is connected to a rear end portion of the inner needle to have an air flow therebetween.

6. The disposable cutting biopsy device of claim 5, wherein
the sliding block comprises a negative pressure transfer pipe that is formed to connect an air flow between the first negative pressure space and the tissue extraction recess.

7. The disposable cutting biopsy device of claim 5, wherein
a first negative pressure valve formed between the first negative pressure space and the negative pressure transfer pipe is
open to transfer a negative pressure formed in the first negative pressure space to the tissue extraction recess when the sliding block moves backward, and closed to prevent a positive pressure formed in the first negative pressure space from being transferred to the tissue extraction recess when the sliding block moves forward, and
a second negative pressure valve formed between the second negative pressure space and an open portion of a rear end of the needle tube, is closed to prevent a positive pressure formed in the second negative pressure space from being transferred to the tissue extraction recess when the sliding block moves backward, and open to transfer a negative pressure formed in the second negative pressure space to the tissue extraction recess when the sliding block moves forward.

8. The disposable cutting biopsy device of claim 1, wherein the sliding block comprises:
a mother block disposed to slide along the outer circumference of the inner needle and having a space formed in a lengthwise direction therein; and
a baby block, to which the outer needle is fixed, assembled to move backward in the mother block,
wherein the baby block comprises a baby block lever to enable assembly and disassembly of the baby block with respect to the mother block.

9. The disposable cutting biopsy device of claim 8, wherein
the mother block and the baby block are disassembled from each other by manipulating the baby block lever, and an extracted tissue sample is collected as the tissue extraction recess of the inner needle is opened by pulling the baby block lever backward.

10. The disposable cutting biopsy device of claim 1, wherein
the loading unit comprises at least one spring connected to a rear portion of the sliding block.

11. The disposable cutting biopsy device of claim 10, wherein
the at least one spring comprises two springs having shapes different from each other.

12. The disposable cutting biopsy device of claim 11, wherein
one spring is loaded by pulling the loading handle, and another spring other than the one spring is loaded by pushing the loading handle.

13. The disposable cutting biopsy device of claim 10, wherein
the loading unit comprises a flange block disposed in the housing and connected to one side of the at least one spring.

14. The disposable cutting biopsy device of claim 10, wherein
the at least one spring comprises at least two springs, and
elasticities of the at least two springs are different from each other.

15. The disposable cutting biopsy device of claim 13, wherein
the at least one spring comprises at least two springs, and
the one spring is loaded by pulling the loading handle, and another spring that is not the one spring is loaded by a compression force generated when the one spring is shot.

16. The disposable cutting biopsy device of claim 13, wherein
the loading unit further comprises:
a first catch device fixing the sliding block; and
a second catch device fixing the flange block.

17. The disposable cutting biopsy device of claim 16, wherein
the at least one spring comprises at least two springs, and
by releasing the first catch device, the sliding block is shot backward by a restoration force of the one spring, and then the sliding block moved backward releases the second catch device and the flange block pushes the sliding block by a restoration force of another spring that is different from the one spring, thereby shooting the sliding block forward.

18. The disposable cutting biopsy device of claim 17, wherein,
after forward shot of the sliding block, the sliding block returns to a fixed position by a restoration force of one of the two springs and is automatically fixed to the first catch device.

19. The disposable cutting biopsy device of claim 18, wherein
the first catch device further comprises:
a shoot button releasing the first catch device;
a first catch protrusion hinge-coupled to the shoot button and protruding into the housing to fix the first sliding block; and
a first restoring spring transferring a restoration force to the shoot button or the first catch protrusion.

20. The disposable cutting biopsy device of claim 18, wherein
the second catch device comprises:
a second catch protrusion fixing the flange block; and
a second restoring spring transferring a restoration force to the second catch protrusion.

21. The disposable cutting biopsy device of claim 13, wherein
a spring accommodation portion is formed in the sliding block or the flange block to accommodate the at least one spring.

22. The disposable cutting biopsy device of claim 1, wherein the sliding block
further comprises at least one O-ring to reduce friction with the sliding space and to keep
sealing during sliding in the housing.

23. The disposable cutting biopsy device of claim 22, wherein
the sliding block comprises a detachment prevention step formed in a front portion and a rear portion of the sliding block to prevent detachment of the O-ring.

24. The disposable cutting biopsy device of claim 1, wherein
the loading handle is disposed to surround a rear portion of the housing.

25. A method of operating the disposable cutting biopsy device according to claim 1, the method comprising:
forming the first negative pressure space in the front portion as the sliding block moves backward, and starting accommodation of a specimen tissue in the tissue extraction recess opened by a backward movement of the outer needle;
applying a negative pressure formed in the first negative pressure space to the tissue extraction recess;
completing the accommodation of the specimen tissue in the tissue extraction recess;
starting cutting of the specimen tissue by the outer needle as the sliding block is shot forward and the second negative pressure space is formed in a rear portion of the housing;
applying a negative pressure formed in the second negative pressure space to the tissue extraction recess; and
completing cutting of the specimen tissue with the outer needle.
